(19) United States
(12) Patent Application Publication
(10) Pub. No.: US 2024/0207339 A1
(43) Pub. Date: Jun. 27, 2024

(54) COMPOSITION COMPRISING A PHYTOCOMPLEX FROM POMEGRANATE AND ITS USES

(71) Applicant: ESSERRE PHARMA SRL, Roma (RM) (IT)

(72) Inventors: Costanza Valentina RICCIONI, Roma (RM) (IT); Amedeo SQUILLACE GRECO, Roma (RM) (IT)

(73) Assignee: ESSERRE PHARMA SRL, Roma (RM) (IT)

(21) Appl. No.: 18/556,692

(22) PCT Filed: Apr. 22, 2022
(86) PCT No.: PCT/EP2022/060782

§ 371 Date: Oct. 23, 2023

(30) Foreign Application Priority Data
Apr. 23, 2021 (IT) 102021000010409

(51) Int. Cl. A61K 36/185; A23L 33/105; A23L 33/155; A61K 31/197; A61K 31/375; A61K 31/4188; A61K 31/4415; A61K 31/455; A61K 31/51; A61K 31/525; A61K 31/592; A61K 31/593; A61K 31/714; A61P 39/06

(52) U.S. Cl. CPC A61K 36/185; A23L 33/105; A23L 33/155; A61K 31/197; A61K 31/375; A61K 31/4188; A61K 31/4415; A61K 31/455; A61K 31/51; A61K 31/525; A61K 31/592; A61K 31/593; A61K 31/714; A61P 39/06

(57) ABSTRACT

A composition containing pomegranate extract and vitamin C and/or vitamin D is disclosed. The composition preferably also includes a B group vitamin or a complex of vitamins of the B group as well as optional substances such as zinc, iron and copper. The pomegranate extract is preferably a dry extract such as one having a total polyphenol content of 15-85% w/w; an ellagitannin content of 5-75% w/w and/or a content of ellagic acid and derivatives thereof of 1-25% w/w. The composition may be included in pharmaceutical, food supplement or drinking products and is useful in activating the immune system, increasing immune defense and/or abilities of the body to recover from psycho-physical stress and/or the prevention and/or treatment of oxidative stress-related conditions or symptoms and/or recovery from tiredness and exhaustion following infection, or counteracting and/or reducing fatigue or tiredness.

### FIELD OF THE INVENTION

The present invention relates to a composition for use as a food supplement and as a functional food, which comprises a particular pomegranate extract, in combination with other functional ingredients such as the vitamins of the B group and/or vitamin C and/or vitamin D. A feature of the present invention is to have a positive effect on the immune defences and on the body's ability to recover from psycho-physical stress. In particular, the associations between pomegranate extract and other ingredients according to the invention have demonstrated a greater antioxidant effect compared to pomegranate extract alone and to the other ingredients taken individually.

### BACKGROUND ART

Nutrition is known to play an important role in the proper functioning of the immune system. On the one hand, malnutrition makes the body more susceptible to infection and, on the other, through proper nutrition the body can introduce substances such as proteins, minerals and vitamins, which contribute to the structuring and maintenance of an efficient immune system. From this point of view, an important role is played by antioxidants, substances taken with food which inactivate ROS (Reactive Oxygen Species) exerting a reducing action and protecting cells from oxidative stress generated in particular during the inflammatory response resulting from an infection (De la Fuente M., Eur J Clin Nutr. 2002). The Mediterranean diet is known to be characterised by foods rich in nutrients and antioxidants, such as polyphenols or vitamins C and E, minerals such as zinc, copper and selenium, obtained in particular from fruit and vegetables typical of the areas of the Mediterranean basin (olive oil—Olea Europaea; plants of the genus Citrus—orange, lemon, grapefruit, bergamot, mandarin; pomegranate—punica granatum; caper—Capparis spinosa, etc.). Nutrition with foods typical of the Mediterranean diet can therefore give support to the body's immune defences (Yahfoufi N, et al, Nutrients, 2018; 1: Carr A C, et al, Nutrients, 2017; Wintergerst E S, et al. Ann Nutr Metab. 2006).

### The Pomegranate

The pomegranate (Punica granatum L.), widely cultivated in the countries of the Mediterranean basin, such as Italy, Spain, Turkey, Egypt, Tunisia and Morocco, is a source of polyphenols such as elligitannins (which mainly include punicalagins and punicalin), ellagic acid, gallic acid and their glycosylated derivatives, anthocyanins. Pomegranates are mainly used in the food industry to obtain juices or jams from the arils, while the peels, which make up about 50% of the fresh fruit by weight, are discarded. It has been reported that the peel has a higher content of polyphenols and a higher antioxidant capacity than pulp, thereby constituting an interesting element for the development of nutraceutical ingredients. There is much evidence that these compounds derived from pomegranate may have protective functions against chronic diseases such as certain cancers, type II diabetes, atherosclerosis and cardiovascular diseases (Landete J., Food Res. Int., 2011; Viuda Martos M., et al, Compr. Rev. Food Sci. Food Saf, 2010). Recent studies also highlight antibacterial and antiviral activities of pomegranate derivatives (Howell A B et al, Evid Based Complem. ALTERNATIVE Med., 2013; Moradi M T et al., Avicenna J Med Biotechnol. 2019; Houston D M J et al., PLoS One, 2017, Malvija S et al., J Food Sci Technol., 2014).

### The B Group Vitamins

Vitamins of the B group are a set of water-soluble vitamins that play an important role in cell metabolism and erythrocyte synthesis. Contained mainly in foodstuffs of animal origin, such as dairy products, meat and eggs, a deficiency of these vitamins can lead to the development of diseases such as beriberi (Vitamin B1 deficiency), macrocytic anaemia (vitamin B12 deficiency), neural tube defects of the unborn child (folic acid deficiency in pregnant women) and epilepsy (vitamin B6 deficiency). The vitamins of the B group contribute to normal energy metabolism, to macronutrient metabolism, to normal functioning of the nervous system and to normal psychological functioning, to maintenance of normal skin, mucous membranes and teguments, to maintenance of normal red blood cells, to iron metabolism, to reduction of tiredness and fatigue, to protection of cells from oxidative stress, and to normal functioning of the immune system (Annex reg EC 432/2012).

### Vitamin C

Vitamin C (ascorbic acid) is a water-soluble vitamin, therefore it cannot be accumulated in the body and must be taken through diet. Vitamin C is characterised by a strong antioxidant power that makes it capable of exercising different functions beneficial to the human body. In particular, it contributes to the protection of cells from oxidative stress, to the normal functioning of the immune system and to the reduction of tiredness and fatigue (Annex reg EC 432/2012). Vitamin C is normally contained in several fresh foods such as oranges, lemons, mandarins, kiwis, strawberries, spinach, broccoli and peppers. Vitamin C deficiencies lead to the development of scurvy, a disease characterised by anaemia, loss of appetite, apathy, muscle pains, gum bleeding and dental fragility.

### Vitamin D

Vitamin D is a fat-soluble vitamin that accumulates in the liver. It occurs in two forms: ergocalciferol (vitamin D2) and cholecalciferol (vitamin D3). Vitamin D is synthesised by our body due to the absorption of sunlight by the skin. It is a regulator of the metabolism of calcium and phosphorus and is involved in the process of calcification of bones. Vitamin D deficiency has negative effects on skeletal tissue, such as the development of rickets in children and osteomalacia in adults. Vitamin D also contributes to the normal functioning of the immune system.

### Fatigue

Fatigue can be defined as a state resulting from physiological and psychological stress that leads to a transient reduction in physical or mental performance (i.e., mental fatigue or physical fatigue) [1]. In general, fatigue is characterized by debilitating periods of exhaustion that interfere with normal activities [2]. The severity and duration of fatigue is variable [3]. Fatigue may result from one or more pathological conditions, but it can also be an entirely physiological condition linked to lifestyle, lack of sleep or bad sleep, stress, family and professional concerns, and environmental factors making it difficult to identify the actual cause [4-6]. One of the most common causes of fatigue is an increase in physical work, leading to a disrupted energy balance due to worked muscle. Muscle fatigue appears quickly and is short-lived. In this case, it has a protective role, warning the subject of the need to rest and recover the body's energy stores. It is traditionally linked to the inhibition of skeletal muscle contraction mechanisms. In fact, the factors involved in fatigue resulting from physical work are numerous and can act at various levels of the chain of command of muscle contraction [7]. In addition to the physiological causes, there are also many pathological conditions associated with fatigue, including autonomic dysfunction, malfunctioning of the hypothalamic-pituitary-adrenal axis, and neuroendocrine dysregulation. Moreover, autoimmune diseases (i.e. systemic lupus erythematosus, multiple sclerosis, type 1 diabetes, celiac disease, chronic fatigue syndrome, and rheumatoid arthritis) can cause fatigue [5].

Fatigue can be classified as prolonged fatigue (or Short-term Fatigue, SF) or chronic fatigue (CFS). SF is a non-pathological condition of fatigue that can be recognized by the subject itself, which consists of persistent or repeated incidence of clinically unexplainable fatigue events. It is an acute form, following on from physical, mental or infectious triggers, and decreases as triggers recede [3]. The duration of the fatigue period may run from 30 days to six months [1]. CFS, without underlying diseases explaining this symptom, is defined as chronic fatigue syndrome\myalgic encephalomyelitis (CFS ME). Differently from SF, subjects with CFS are not usually able to restore an adequate energy level, even after a rest or the elimination of the causes. Fatigue can be considered CFS if its duration is longer than six months [8].

Short periods of fatigue occur in 10-33% of total population. SF is an universal symptom expressed by the majority of the population occurring in about 5-8% of the general population, while CFS occurs in 3-4% of general population. Fatigue is debilitating and often has significant economic consequences. A Dutch study found that 21.5% of adult workers reported prolonged fatigue, which may lead to lower productivity at work [4]. There are also affective consequences of fatigue, reported as mood swings and lack of motivation and vitality [6]. Many studies reported that fatigue elicited by physical training translates into worsened sport performance. For instance, cross-country skiers had reduced double poling performance after a 25 min trunk fatiguing exercise sequence, compared with a 25 min rest [9]. Fatigued ballet dancers showed reduced movement control, which may increase the risk of injury [10]. Interestingly, impaired performance appeared to be associated with not only physical and muscular sports, but also includes mental factors. For example, table tennis players hit the ball with lower attention and speed after completion of a 90 min cognitive task [11]. In addition, many studies show that in Europe overtiredness at the wheel is the leading cause of death on the roads, and that accidents related to drowsiness are more serious and deadly than those in which sleepiness is not involved [12].

No pharmacological or alternative therapy has been proven effective to date for subjects with CFS [8]. Although some treatments were tested, such as cognitive behavioral therapies and exercise, both seem to show many limitations. For fatigue caused by pathologies like multiple sclerosis, Modafinil, Amantadine and Methylphenidate are used, but according to certain studies these do not have effects much higher than placebo [13].

Due to the unavailability of effective remedies that attenuate fatigue and provide consumer satisfaction, there is the need for a food or nutraceutical composition that can help, among others, to fight CFS and SF.

### SUMMARY OF THE INVENTION

The authors surprisingly found that the use of a pomegranate extract, in combination with other functional ingredients such as vitamins of the B group and/or vitamin C and/or vitamin D, has potent antioxidant effects in vitro.

The antioxidant effect of the composition suggests for it a health effect on humans that supports the immune defences, reduction of the risk of infection, improvement of the body's response to stress or insult, reduction of tiredness and fatigue, improvement of physical and cognitive performance.

In particular, a combination of pomegranate extract and vitamin C, and preferably the vitamins of the B group, or a combination of pomegranate extract and vitamin D, exerts a synergistic action, increasing the antioxidant effects. The combinations according to the invention have a greater antioxidant effect than the single effect of pomegranate extract alone or of vitamins B, vitamin C, vitamin D alone.

In particular, the tested compositions have a greater antioxidant effect than the sum of the antioxidant effects of the main ingredients, with significant antioxidant activity (pomegranate, the vitamins of the B group and vitamin C, or pomegranate and vitamin D).

The present inventors also found that a pomegranate extract, B vitamins and vitamin C—based food supplement help to fight SF, measured through the administration of questionnaires assessing fatigue level before and after the daily intake of the supplement for one month, and additionally assessing if this food supplement is well tolerated.

In addition, the metabolic profile of the whole fruit pomegranate extract used as an ingredient in the present food supplement, was analyzed using the latest generation of LC-MS instrumentation, to deepen the knowledge on the chemical composition of the phytocomplex present in this food supplement.

Therefore, the object of the invention is a composition comprising pomegranate extract and at least one substance selected from vitamin C or vitamin D.

Preferably, the composition of the invention comprises pomegranate extract and at least one substance selected from vitamin C or vitamin D as active ingredients.

Preferably the composition of the invention comprises pomegranate extract, vitamin C and at least one vitamin of B group.

Preferably, the composition of the invention consists of pomegranate extract, vitamin C, at least one vitamin of B group and optionally at least one substance selected from the group consisting of: zinc, iron and copper.

Preferably, the composition of the invention consists of pomegranate extract, vitamin C, at least one vitamin of B group.

Preferably the composition of the invention comprises pomegranate extract and vitamin D or pomegranate extract, vitamin C and vitamin D.

Preferably the composition of the invention consists of:
pomegranate extract and vitamin D or
pomegranate extract, vitamin C and vitamin D
and optionally at least one substance selected from the group consisting of: zinc, iron and copper.

Preferably the composition of the invention consists of:
pomegranate extract and vitamin D or
pomegranate extract, vitamin C and vitamin D.

Preferably the pomegranate extract is present in an amount from 100 mg to 600 mg.

Preferably the pomegranate extract is a dry extract.

Preferably the pomegranate extract is characterised by:
a total polyphenol content comprised between 15% w/w and 45% w/w, preferably 15% or 20%, and/or
an elligitannin content comprised between 5% and 15% w/w, preferably from 7% to 10%, and/or
an ellagic acid and derivative content comprised between 1% w/w and 15% w/w, preferably not less than 3%.

Preferably the pomegranate extract is obtained by at least one extraction of pomegranate fruit, peel, or parts thereof with a solvent selected from: ethanol, water, ethanol-water mixtures.

Preferably the pomegranate extract derives from pomegranate fruit or peel or parts thereof.

Preferably the pomegranate extract is characterized by:
a total polyphenol content comprised between 15% w/w and 85% w/w, preferably between 15% w/w and 45% w/w, preferably 15 or 20%, and/or
an ellagitannin content comprised between 5 and 75% w/w, preferably between 5 and 15% w/w, preferably comprised between 7 and 10%, and/or
a content of ellagic acid and derivatives comprised between 1% w/w and 25% w/w, preferably between 1% w/w and 15% w/w, preferably not less than 3%.

Preferably the composition of the invention comprises pomegranate extract, complex of vitamins of B group and vitamin C.

Preferably the complex of vitamins of the B group comprises at least one vitamin selected from the group consisting of: Vitamin B1, vitamin B2, vitamin B6, vitamin B12, pantothenic acid, niacin, biotin and folate.

Preferably the complex of vitamins of the B group comprises: Vitamin B1 and/or vitamin B2 and/or vitamin B6 and/or vitamin B12 and/or pantothenic acid and/or vitamin B3 and/or biotin and/or folate.

Preferably the complex of vitamins of the B group comprises: Vitamin B1, vitamin B2, vitamin B6, vitamin B12, pantothenic acid, vitamin B3, biotin and folate.

Preferably the composition of the invention comprises in percentage referred to the total weight of the composition:
pomegranate extract 5-80%, preferably about 31.2% or 25%,
complex of vitamins of B group 1-10%, preferably about 2.4% or 4%,
Vitamin C 5-30%, preferably about 12.5% or 8%.

Preferably, the composition includes a percentage referring to the total weight of the composition: pomegranate extract (5-80%, preferably at least 15%, preferably about 31.2% or 25%) and/or the complex of vitamins of the B group (1-10%, preferably about 2.4% or 4%) and/or vitamin C (5-30%, preferably about 12.5% or 8%).

Preferably the composition of the invention comprises pomegranate extract and vitamin D, preferably said composition comprises in percentage referred to the total weight of the composition: pomegranate extract 5-20%, preferably about 6%, and vitamin D 0.0001-0.10%, preferably about 0.0005%.

Preferably the composition of the invention comprises pomegranate extract, vitamin D and vitamin C, preferably said composition comprises in percentage referred to the total weight of the composition: pomegranate extract 5-50%, preferably about 20%, vitamin D 0.0001-0.10%, preferably about 0.001%, vitamin C 5-30%, preferably about 2.6%.

Preferably the composition comprises in the w/w percentages with respect to the total of the active ingredients: pomegranate extract (40-80%, about 68%, preferably 67.7%), the complex of vitamins of the B group (1-10%, preferably about 5%), vitamin C (5-40%, preferably about 27%).

Preferably the composition comprises in the w/w percentages with respect to the total of the active ingredients: pomegranate extract (40-80%, preferably about 68%, preferably 67.7%), the complex of vitamins of the B group (1-10%, preferably about 5%), vitamin C (5-40%, preferably about 27%).

Preferably the composition comprises in the w/w percentages with respect to the total of the active ingredients: pomegranate extract about 68%, the complex of vitamins of the B group about 5%, vitamin C about 27%.

Preferably the composition comprises pomegranate extract of at least 40% or 55% in the w/w percentages with respect to the total of the active ingredients.

Preferably the composition comprises pomegranate extract and vitamin D, preferably said composition comprises in percentage terms with reference to the total weight of the composition: pomegranate extract (5-20%, preferably about 6%) and/or vitamin D (0.0001-0.10%, preferably about 0.0005%).

Preferably the composition comprises pomegranate extract and vitamin D, preferably said composition comprises in percentage terms with reference to the total weight of the composition: pomegranate extract (5-20%, preferably about 6%) and vitamin D (0.0001-0.10%, preferably about 0.0005%).

Preferably the composition comprises, in w/w percentages with respect to the total of the active ingredients: pomegranate extract (70-99.999%, preferably about 99.99%) and vitamin D (0.0001-30%, preferably about 0.01%).

Preferably the composition comprises, in w/w percentages with respect to the total of the active ingredients: pomegranate extract about 99.99% and vitamin D about 0.01%.

Preferably the composition comprises, in w/w percentages with respect to the total of the active ingredients: pomegranate extract about 88% and vitamin D about 12%.

Preferably the composition comprises pomegranate extract, vitamin D and vitamin C, preferably said composition comprises in percentage terms with reference to the total weight of the composition: pomegranate extract (5-50%, preferably about 20%) and/or vitamin D (0.0001-0.10%, preferably about 0.001%) and/or vitamin C (5-30%, preferably about 2.6%).

Preferably the composition comprises pomegranate extract, vitamin D and vitamin C, preferably said composition comprises in percentage terms with reference to the total weight of the composition: pomegranate extract (5-50%, preferably about 20%), vitamin D (0.0001-0.10%, preferably about 0.001%) and vitamin C (5-30%, preferably about 2.6%).

Preferably in the composition:
i. the pomegranate extract present in an amount from 100 mg to 600 mg, preferably 250 mg or 300 mg or 350 mg 500 mg or 600 mg and/or
ii. the vitamins of the B group present in the following amounts:
   vitamin B1: 1.1 mg to 25 mg, preferably 5.5 mg and/or
   vitamin B2: 1.4 mg to 25 mg, preferably 7 mg and/or
   vitamin B6: 1.4 mg to 10 mg, preferably 4 mg and/or
   vitamin B12: from 2.5 mcg (micrograms) to 1000 mcg, preferably 0.0125 mg and/or
   niacin: 16 mg to 54 mg, preferably 16 mg and/or
   pantothenic acid: 6 mg to 18 mg, preferably 6 mg and/or
   biotin: from 50 mcg to 450 mcg, preferably 0.05 mg and/or
   folic acid or folate: from 200 mcg to 400 mcg, preferably 0.2 mg and/or
iii. vitamin C present in an amount of from 40 mg to 1000 mg, preferably 80 mg or 100 mg or 200 mg and/or
iv. vitamin D present in an amount of from 5 mcg to 50 mcg, preferably 5 mcg or 10 mcg or 25 mcg or 50 mcg.

Preferably in the composition:
i. pomegranate extract is present in an amount from 100 mg to 600 mg, preferably 150, 250, 300, 350, 500 or 600 mg and/or
ii. B group vitamins are present in the following quantities:
   Vitamin B1: from 1.1 mg to 25 mg, preferably 5.5 mg and/or
   Vitamin B2: from 1.4 mg to 25 mg, preferably 7 mg and/or
   Vitamin B6: from 1.4 mg to 10 mg, preferably 4 mg and/or
   Vitamin B12: from 2.5 mcg (micrograms) to 1000 mcg, preferably 0.0125 mg and/or
   Niacin: 16 mg to 54 mg, preferably 16 mg and/or
   Pantothenic acid: 6 mg to 18 mg, preferably 6 mg and/or
   Biotin: from 50 mcg to 450 mcg, preferably 0.05 mg and/or
   folic acid: from 200 mcg to 400 mcg, preferably 0.2 mg and/or
iii. vitamin C is present in an amount of from 40 to 1000 mg, preferably 80 or 100 or 200 mg and/or
iv. vitamin D is present in an amount of 5 to 50 mcg, preferably 5 or 10 or 25 or 50 mcg.

Preferably in the composition:
i. pomegranate extract is present in an amount from 100 mg to 600 mg, preferably 150 or 250 or 300 or 350 or 500 or 600 mg and
ii. B group vitamins are present in the following quantities:
   Vitamin B1: from 1.1 mg to 25 mg, preferably 5.5 mg and
   Vitamin B2: from 1.4 mg to 25 mg, preferably 7 mg and
   Vitamin B6: from 1.4 mg to 10 mg, preferably 4 mg and
   Vitamin B12: from 2.5 mcg (micrograms) to 1000 mcg, preferably 0.0125 mg and
   Niacin: 16 mg to 54 mg, preferably 16 mg and
   Pantothenic acid: 6 mg to 18 mg, preferably 6 mg and
   Biotin: from 50 mcg to 450 mcg, preferably 0.05 mg and
   folic acid: from 200 mcg to 400 mcg, preferably 0.2 mg and
iii. vitamin C is present in an amount of from 40 to 1000 mg, preferably 80 or 100 or 200 mg.

Preferably in the composition of the invention:
pomegranate extract is present in an amount from 100 mg to 600 mg, preferably 150 or 250 or 300 or 350 or 500 or 600 mg and
vitamin C is present in an amount of from 40 to 1000 mg, preferably 80 or 100 or 200 mg and
vitamin D is present in an amount of 5 to 50 mcg, preferably 5 or 10 or 25 or 50 mcg.

Preferably in the composition of the invention:
pomegranate extract is present in an amount from 100 mg to 600 mg, preferably 150 or 250 or 300 or 350 or 500 or 600 mg and
vitamin D is present in an amount of 5 to 50 mcg, preferably 5 or 10 or 25 or 50 mcg.

In the composition according to the invention, preferably the pomegranate extract, vitamin C and vitamin D and the vitamins of the B group are the only substances present in the composition which are active in activating the immune system and/or in increasing the immune defenses and/or the body's ability to recover from psycho-physical stress, in strengthening the immune system, reducing the period and intensity of colds and flu infections, preventing colds and flu and/or fighting colds and influenza, and/or preventing and/or treating upper respiratory tract infections, influenza virus infection, in convalescence after a seasonal illness or a surgery, and/or in the prevention and/or treatment of diseases or symptoms related to oxidative stress and/or in recovering from tiredness and exhaustion following an infection, or other cause and/or in countering and/or reducing fatigue or tiredness, preferably chronic fatigue.

Therefore, in the context of the present invention the pomegranate extract, vitamin C and vitamin D and the vitamins of the B group may be defined as active ingredients.

A further object of the invention is a pharmaceutical composition comprising the composition according to the invention and at least one pharmaceutically acceptable excipient and/or vehicle. Another object of the invention is a food supplement or product or drinking product comprising the composition according to the invention.

The composition or pharmaceutical composition or the food supplement or product or drinking product according to the invention are preferably for medical use, preferably for use in activating the immune system or in increasing the immune defenses and/or abilities of the body to recover from psycho-physical stress, preferably for use in strengthening the immune system, reducing the period and intensity of colds and flu infections, preventing colds and flu and/or fighting colds and flu, and/or for use in the prevention and/or treatment of upper respiratory tract infections, influenza virus infection, in convalescence after seasonal illness or surgery, and/or for use in the prevention and/or treatment of oxidative stress-related conditions or symptoms and/or in recovery from tiredness and exhaustion following an infection, or other cause and/or in counteracting and/or reducing fatigue or tiredness, preferably chronic fatigue.

More preferably the composition or the pharmaceutical composition or the food supplement or product or drinking product according the invention is for use in counteracting and/or reducing fatigue or tiredness, preferably chronic fatigue.

Another object of the invention is a non-therapeutic use of the composition or of the food supplement or product or drinking product according to the invention in the nutraceutical sector or as a basic ingredient in preparations of supplements or drugs and/or as an antioxidant agent.

A further subject matter of the invention is the non-therapeutic use of the composition or supplement according to the invention in the nutraceutical sector or as a basic ingredient in preparations of supplements or drugs and/or as an antioxidant agent.

The amounts described herein are preferably intended as daily quantities.

Another subject matter of the invention is a composition comprising the composition as defined above and at least one excipient and/or vehicle.

A further subject matter of the invention is a food or nutraceutical supplement comprising the composition as defined above.

The composition as defined above or the pharmaceutical composition as defined above is preferably for medical use, preferably for use in increasing the immune defences and capacities of the body to recover after psycho-physical stress, preferably for use in the prevention and/or treatment of upper respiratory tract infections, influenza, and in convalescence after seasonal illness or surgery.

The composition as defined above or the dietary or nutraceutical supplement as defined above may be used to increase the immune defences and the resilience of the organism after psycho-physical stress, preferably to prevent and treat upper respiratory tract infections and flu, and during convalescence after a seasonal disease or surgery.

A further subject matter of the invention is the non-therapeutic use of the composition or supplement as defined above in the nutraceutical sector or as a basic ingredient in supplement or drug preparations and/or as an antioxidant agent.

### DETAILED DESCRIPTION OF THE INVENTION

In the present invention an “extract” is obtained by an extraction step wherein a solvent is added to a matrix (liquid or solid) to extract only the molecules of interest which will then result concentrated in the solvent of extractions compared to the starting matrix.

Preferably, the pomegranate extract of the present invention is extracted by adding a solvent (water, alcohol, or water/alcohol) to a pomegranate matrix, thus obtaining a liquid extract in which the molecules of interest are concentrated. The liquid extract is then preferably filtrated and/or concentrated (liquid concentrated extract). The extract may be atomized to become a standardized dry extract. The pomegranate extract included in the invention is preferably an extract from pomegranate peel, or from pomegranate peel and juice (obtained, for example, by pressing the arils) characterised by:
a total polyphenol content comprised between 15% w/w and 45% w/w, preferably 15% or 20%, and/or
an elligitannin content comprised between from 5% and 15% w/w, preferably comprised between 7% and 10%, and/or
an ellagic acid and derivative content comprised between 1% w/w and 15% w/w, preferably not less than 3%.

Preferably the pomegranate extract presents at least one of the compounds mentioned in Table 1 and/or Table 5.

The pomegranate extract with the characteristics described above may be obtained from dried and ground pomegranate fruits mixed with a hydroalcoholic solution (food grade) and then filtered. The filtrate is then concentrated and subjected to drying process by a spray drying to obtain the standardised extract, i.e. an extract that contains a titre of active ingredients that is always the same. The filtrate can more preferably be obtained using the procedure described in Sorrenti et al., Frontiers in Microbiology, 2019, herein incorporated by reference.

Preferably the pomegranate extract is a dry extract.

Preferably the vitamin D is selected from the group consisting of: vitamin D3 (cholecalciferol), vitamin D2 (ergocalciferol), 25 (OH) vitamin D (calcidiol) and 1.25 dihydroxy-vitamin D (calcitriol). More preferably, the vitamin D is vitamin D3. Preferably, the vitamins of the B complex are: vitamin B1, vitamin B2, vitamin B6, vitamin B12, niacin, pantothenic acid, biotin and folic acid.

Preferably, the pomegranate extract and vitamin C are in a w/w ratio of 10:1, 9:1, 8:1, 7:1, 6:1 5:1, 4:1, 3:1, 2.5:1, 2:1, 1:1, 1:2, 1:3, 1:4, 1:5, 1:6, 1:7, 1:8, 1:9, 1:10, preferably 2.5:1.

Preferably, the pomegranate extract and the vitamin B complex are in a ratio of 20:1, 13:1, 12, 5:1, 12:1, 10:1, 5:1, 2:1, preferably 12, 5:1.

Preferably, the pomegranate extract and vitamin D are in a ratio of 50,000:1, 40,000:1, 30,000:1, 25,000:1, 20,000:1, 15,000:1, 12,000:1, 10,000:1, 6,000:1, 5,000:1, preferably 12,000:1.

Preferably, the excipient and/or diluent is selected from the group consisting of: calcium phosphate, dicalcium phosphate, microcrystalline cellulose, magnesium stearate, silicon dioxide, sucrose, gum arabic, corn starch, medium chain triglycerides, tricalcium phosphate, cross-linked sodium carboxymethylcellulose, hydroxypropylmethylcellulose, polyethylene glycol, titanium dioxide, polyvinylpyrrolidone, talc, erythritol, xylitol, steviol glycosides and sucralose.

Preferably, the composition as defined above is administered orally. Preferably, said composition is administered once or twice per day. Preferably, said composition is in the form of a tablet, a hard capsule, a soft gel capsule, a powder, a syrup, a cachet, a lozenge, a bar, or a liquid or semi-liquid.

A further subject matter of the present invention is a food supplement or a food product or a drinking product comprising the composition described above and at least one excipient or diluent and, optionally, a further agent.

Preferably, said food supplement or food product or drinking product is administered orally. Preferably, said food supplement or product or drinking product is administered once or twice per day. Preferably, said food supplement or food product or drinking product is in the form of a tablet, a hard capsule, a soft gel capsule, a powder, a syrup, a cachet, a lozenge, a tablet, a pill, a food supplement, an edible bar, or an edible snack. In particular, said supplement may be any type of food supplement.

It is another object of the invention a method of treating any of the diseases, symptoms, conditions herein mentioned, the method comprising administering to a subject in need thereof a therapeutically effective amount of a composition as disclosed herein.

It is another object of the invention the use of the composition or the pharmaceutical composition or the food supplement or product or drinking product as defined herein for activating the immune system or for increasing the immune defenses and/or abilities of the body to recover from psycho-physical stress, preferably for strengthening the immune system, reducing the period and intensity of colds and flu infections, preventing colds and flu and/or fighting colds and flu, and/or for prevention and/or treatment of upper respiratory tract infections, influenza virus infection, in convalescence after seasonal illness or surgery, and/or for prevention and/or treatment of oxidative stress-related conditions or symptoms and/or in recovery from tiredness and exhaustion following an infection, or other cause and/or for counteracting and/or reducing fatigue or tiredness, preferably chronic fatigue.

The following provides definitions of various terms and expressions used in the present application to disclose the invention. It is to be understood that the definitions of terms and expressions employed in more than one aspect or embodiment of the invention equally apply to any aspect or embodiment of the invention disclosed herein in which those terms and expressions appear. This also applies regardless of where (i.e. in which section in the present application) these terms are defined or discussed.

Furthermore, although the present application mentions separate embodiments, it is to be understood that any embodiment and the features therein may be freely combined with any other embodiment and features therein, even in the absence of an explicit statement to that effect. Such combinations of one embodiment with another, or of one or more features of any embodiment with one or more features of any other embodiment, are thus part of the disclosure of the present application as filed, as understood by a person skilled in the art.

The term “comprising” as used herein has the broad common meaning of “including,” “covering,” “containing,” or “consisting of” It includes the element or elements that are explicitly recited and also allows, but does not require, the presence of another or other elements that are not recited. In addition to this broad meaning, as used herein, the term “comprising” also covers the limiting meaning of “consisting of”, according to which only the element or elements explicitly recited and no other are present. In addition, the term “comprising” also includes the meaning of “essentially consisting of”, which denotes that there may be one or more other elements in addition to those explicitly recited, provided that the additional element or elements present do not alter the technical effect obtained by the element or elements explicitly recited.

As used herein, the term “subject” refers to a mammal, preferably a human.

The term “vitamin D” includes and describes the substance with the chemical formulas C₂₇H₄₄O=vitamin D3 (cholecalciferol), C₂₈H₄₄O=vitamin D2 (ergocalciferol), C₂₇H₄₄O₂=25 (OH) vitamin D (calcidiol), C₂₇H₄₄O₃=1.25 dihydroxy-vitamin D (calcitriol). Preferably, vitamin D refers to vitamin D3.

Preferably, vitamin D comprises from 0.75 μg (25 IU) to 50 μg (2000 IU) of vitamin D/g. Equally preferably, 1 IU corresponds to 0.025 μg of vitamin D.

In the context of the present invention the term folic acid and folate are interchangeable.

The term folic acid or folate may also include any form of folic acid.

The term vitamin includes any form of the vitamin, including any salt.

The vitamins referred to in this invention comprise their corresponding analogues or derivatives, for example (and as not limiting examples), vitamin B1 means thiamine and analogs or derivatives thereof, vitamin B2 denotes riboflavin and analogs or derivatives thereof, vitamin B3 refers to niacin, nicotinic acid and analogs or derivatives thereof, vitamin B5 means pantothenic acid and analogs or derivatives thereof, vitamin B6 means pyridoxine and analogs or derivatives thereof, vitamin B7 is biotin and analogues or derivatives thereof; vitamin B9 means folic acid and analogs or derivatives thereof, vitamin B12 means cyanocobalamine and analogs or derivatives thereof, vitamin C means ascorbic acid and analogs or derivatives thereof, and so on.

The term vitamin C as used herein encompasses and describes the substance with the chemical formula C₆H₈O₆ in the form of 1-ascorbic acid or sodium L-ascorbate, or calcium L-ascorbate, or potassium L-ascorbate or L-ascorbyl 6-palmitate or sodium ascorbate (L-sodium ascorbate), magnesium ascorbyl phosphate, L-Ascorbate-polyphosphate.

An embodiment of the present invention comprises a combination of pomegranate extract and vitamin C.

In a preferred embodiment, the present invention comprises a combination comprising pomegranate extract, vitamin C and at least one B vitamin. More preferably, the combination comprises pomegranate extract, vitamin C, vitamin B1, vitamin B2, vitamin B6, vitamin B12, pantothenic acid, niacin, biotin and folate and optionally copper and/or zinc and/or iron.

In a preferred embodiment, the present invention comprises a combination comprising pomegranate extract and vitamin D. In a preferred embodiment, the present invention comprises a combination comprising pomegranate extract and vitamin D and vitamin C.

In preferred embodiments, the present invention refers to the following compositions:
1. Pomegranate extract 500 mg
   Vitamin C 200 mg
   Vitamin B1 5.5 mg
   Vitamin B2 7 mg
   Vitamin B6 4 mg
   Vitamin B12 12.5 μg
   Pantothenic acid 6 mg
   Niacin 16 mg
   Biotin 50 μg
   Folate 200 μg,
2. Pomegranate extract 250 mg
   Vitamin C 80 mg
   Niacin 16 mg
   Vitamin B2 7 mg
   Pantothenic acid 6 mg
   Vitamin B1 5.5 mg
   Vitamin B6 4 mg
   Folate 0.2 mg
   Biotin 0.05 mg
   Vitamin B12 0.0125 mg
   Copper 2 mg
   Zinc 10 mg
   Iron 30 mg
3. Pomegranate extract 300 mg
   Vitamin D 0.025 mg
4. Pomegranate extract 600 mg
   Vitamin D 0.050 mg
   Vitamin C 80 mg
5. Pomegranate extract 150 mg
   Vitamin D 50 mcg
6. Pomegranate extract 350 mg
   Vitamin D 50 mcg

Preferably, the composition of the invention is in unit doses, such as a stick, and comprises:

| | | |
|---|---|---|
| Pomegranate dry extract titrated at 20% in | 500 | mg |
| polyphenols | | |
| Vitamin C | 200 | mg |
| Niacin | 16 | mg |
| Vitamin B2 | 7 | mg |
| Pantothenic acid | 6 | mg |
| Vitamin B1 | 5.5 | mg |
| Vitamin B6 | 4 | mg |
| Folate | 0.2 | mg |
| Biotin | 0.05 | mg |
| Vitamin B12 | 0.0125 | mg |

Preferably, the composition of the invention comprises the following substances in the following w/w percentages with respect to the total of active ingredients:

| | |
|---|---|
| Pomegranate dry extract titrated at 20% in | 67.7% |
| polyphenols | |
| Vitamin C | 27% |
| Niacin | 2.16% |
| Vitamin B2 | 0.94% |
| Pantothenic acid | 0.81% |
| Vitamin B1 | 0.74% |
| Vitamin B6 | 0.54% |
| Folate | 0.027% |
| Biotin | 0.006% |
| Vitamin B12 | 0.001% |

Preferably, the composition of the invention comprises the following substances in the following amounts per 100 g of composition:

| | | |
|---|---|---|
| Pomegranate dry extract | 31.25 | g |
| Vitamin C | 12,500 | mg |
| Niacin | 1,000 | mg |
| Vitamin B2 | 437.5 | mg |
| Pantothenic acid | 375 | mg |
| Vitamin B1 | 343.75 | mg |
| Vitamin B6 | 250 | mg |
| Folate | 12,500 | μg |
| Biotin | 3,125 | μg |

| | |
|---|---|
| Vitamin B12 | 781.25 |

Preferably the pomegranate extract is a dry fruit extract titrated at 20% in polyphenols.

Any drying process known in the art may be used to dry the pomegranate extract, such as osmotic dehydration, lyophilisation, inert gas drying, air drying or vacuum drying.

The compositions defined above are, for example, in the form of an oral powder or granulate for oral solution in a sachet.

The composition according to the invention may further include at least one substance selected from the group consisting of: zinc, iron, copper, extract of Citrus aurantium var amara L, Citrus aurantium var dulcis Hayne, Citrus x bergamia risso et poit, citrus limon L. Osbeck, Citrus maxima Merr., Citrus medica L., Citrus myrtifolia Raf, Citrus nobilis, Citrus paradisi, Citrus reticulata, Citrus sinensis osbeck, Citrus japonica, propolis extract, pineapple extract comosus, bromelain, opuntia Ficus indica (L.) Mill extract.

Preferably the zinc is present in an amount of from 5 mg to 15 mg, preferably 10 mg, and/or the iron is present in an amount of from 5 mg to 50 mg, preferably 30 mg, and/or the copper is present in an amount of from 1 mg to 10 mg, preferably 2 mg.

Agents of the present invention may exist and be used according to the invention in any salt, solvate, stereoisomeric, zwitterionic and/or isotopic form.

The salts of the agents of the present invention are preferably pharmaceutically acceptable. Proper pharmaceutically acceptable salts include conventional non-toxic salts obtained by salification of an agent of the present invention with inorganic acids (e.g. hydrochloric, bromidic, sulfuric or phosphoric acids) or with organic acids (e.g. acetic, propionic, succinic, benzoic, sulfanilic, 2-acetoxybenzoic, cinnamic, mandelic, salicylic, glycolic, lactic, oxalic, malic, maleic, malonic, fumaric, tartaric, citric, p-toluenesulfonic, methanesulfonic, ethanesulfonic or naphthalene sulfonic acids). Proper pharmaceutically suitable salts are described for example in Berge S. M. et al., J. Pharm. Sci. 1977, 66, 1-19; Gould P. L. Int. J. Pharm 1986, 33, 201-217; Bighley et al. Encyclopedia of Pharmaceutical Technology, Marcel Dekker Inc, New York 1996, Volume 13, pages 453-497; and Remington “The Science and Practice of Pharmacy,” Lippincott Williams & Wilkins, 2000. In addition, the agents of the present invention can exist in unsolvated as well as solvated forms, with pharmaceutically acceptable solvents such as water, EtOH, and the like.

The invention also includes the use of all suitable isotopic variations of agents of the present invention. An “isotopic variant” of an agent of the invention is defined as one in which at least one atom is replaced by an atom having the same atomic number but an atomic mass which is different from the atomic mass usually present in nature. Examples of isotopes that can be incorporated into agents of the invention include respectively isotopes such as ²H, ³H, ¹³C, ¹⁴C, ¹⁵N, ¹⁷O, ¹⁸O, ³¹P, ³²P, ³⁵S, ¹⁸F, and ³⁶Cl. Certain isotopic variations of the invention, for example, those into which a radioactive isotope such as ³H or ¹⁴C is incorporated, are useful in studies of the distribution of substrates and/or drugs in tissues. Furthermore, replacement with isotopes such as ²H deuterium can lead to therapeutic benefits resulting from increased metabolic stability. Isotopic variations of the agents of the invention can generally be prepared by conventional procedures such as with the illustrative methods or with the preparations disclosed in the examples below using suitable isotopic variations of suitable reagents.

A given treatment regimen may comprise multiple and repeated co-administrations of the composition according to the invention for a predetermined period of time, for example 1 month, 2 months, 3 months, 4 months, 5 months, 6 months, or more, for a predetermined period of at least of time 4 months being preferred. Co-administration can be repeated multiple times per day, for example once, twice, three times, four times, five times or more, a repetition of about once per day being preferred. In other words, co-administration is repeated for a predetermined amount of time, and the sum of the co-administration instances in this predetermined amount of time constitutes the prophylactic or therapeutic regimen.

As used herein, the term “composition” or “combination” includes and describes any physical entity comprising or consisting of or consisting essentially of the respective recited substances, for example comprising or consisting of or essentially consisting of pomegranate extract and/or vitamin D and/or C. The physical form of the composition is not limited. For example, the term “composition” comprises and describes a powder in which each of the recited substances is present as a powder. As a further example, the term “composition” also comprises and describes a liquid solution in which the recited substances are present in solubilised form. As a further example, the term “composition” also encompasses and describes an emulsion in which the recited substances are present. As a further example, the term “composition” also encompasses and describes a suspension in which the recited substances are present. As a further example, the term “composition” also includes and describes mixtures in which the pomegranate extract is in one form, for example a solid such as a powder, while the vitamin D is in another form, for example a liquid. In particular, the term “composition” may be a “pharmaceutical composition” as defined below and may be formulated for a desired route of administration. As used and described herein, the term “composition” may further be a composition suitable for oral delivery, for example in the form of a tablet, including, but not limited to, an effervescent tablet or a multilayer tablet, a powder, for example in the form of a sachet, a hard capsule, a soft gel capsule, a syrup, a cachet, a bar, a pastille, for example a soft pastille, a balm, or a liquid preparation. In some particularly preferred embodiments of the invention, the “composition” may be in the form of a soft gel capsule or in the form of a powder. The term “composition” may also be a composition suitable for non-oral delivery, for example in the form of a suppository, a tablet, a hard capsule, a soft gel capsule, a cream, a gel, a patch or a liquid. Further dose forms of the composition as well as the indicated routes of administration are set out below. Preferably the composition or pharmaceutical composition of the invention is in a unit dose such as a stick, a tablet, a sachet, a vial, . . . .

Compositions of the invention, including pharmaceutical compositions, may further comprise at least one pharmaceutically acceptable ingredient. As used herein, the term “food product” refers to an ingestible substance that, at the temperature at which it is properly stored and ingested, is in a solid or semi-solid form, and that will be chewed before being swallowed. As used herein, the term “drinking product” refers to an ingestible substance which, at the temperature at which it is properly stored and ingested, is in a free-flowing liquid form, and which will not be chewed before being swallowed. Examples of such food products include a food bar, such as a chocolate bar, a granola bar, an ice cream bar, or an energy bar; a chewing gum; a candy bar; a mint; a yoghurt; an edible gel; a ready meal, such as a frozen-dried ready meal; a spreadable cream; a pudding; or a processed fruit based product such as a fruit roll or a fruit skewer. Examples of such drinking products include fruit juice or drinks containing fruit juice, dairy-based drinks, for example drinks containing milk or drinks containing buttermilk, drinks containing whey and drinks containing yoghurt, energy drinks, soft drinks, flavoured water drinks, etc. Also included in the present invention is an article of manufacturing comprising packaging material comprising inside a dose form effective for activating the immune system of a person, and packaging material comprising a label indicating that the composition can be used to activate the immune system and wherein said composition is as described above.

As used herein, the term “immune system activation” means, for example, improvements in all types of situations, in which it is assumed that a person's immune system will perform at a higher level, including strengthening that person's immune system; reducing the period and intensity of cold and flu infections in that person; preventing cold and flu in that person; and ameliorating cold and flu symptoms in that person. The following provides several examples and figures illustrating various embodiments of the present invention and the technical effects and advantages that it affords. It should be understood that the following examples are presented by way of illustration only and do not limit the invention claimed. The person skilled in the art will in fact be readily able to realise other embodiments within the spirit and purpose of the claimed invention, while obtaining the technical advantages disclosed.

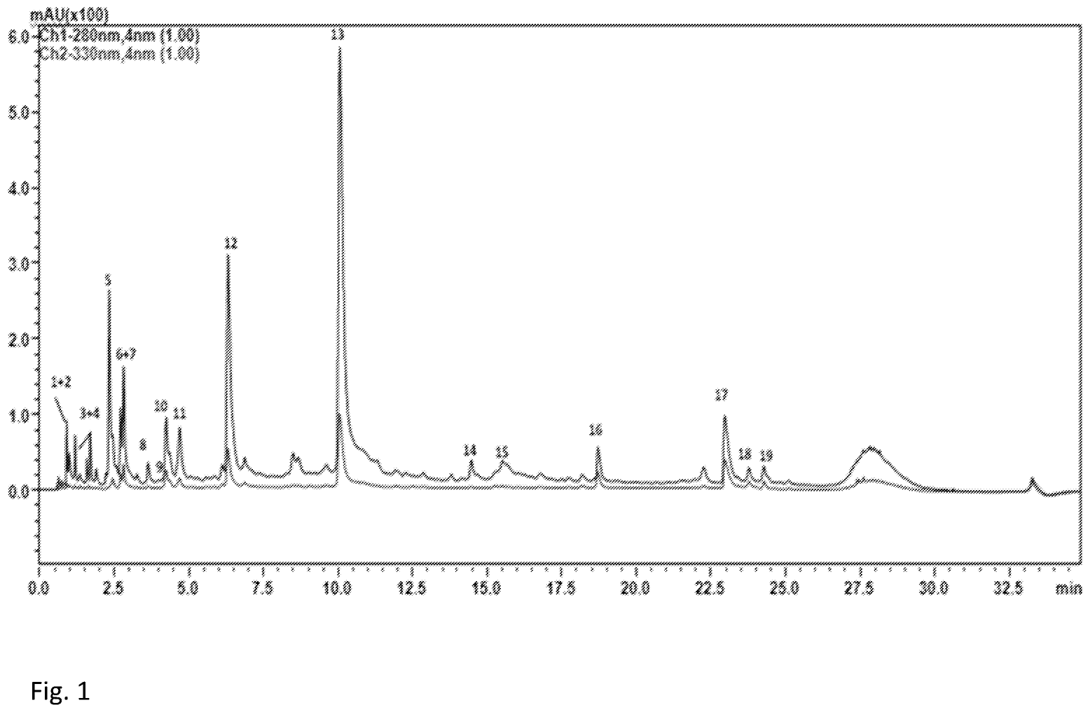

FIG. 1. RP-UHPLC-UV-IRMS analysis of pomegranate dry extract (PunicaPLUS®)

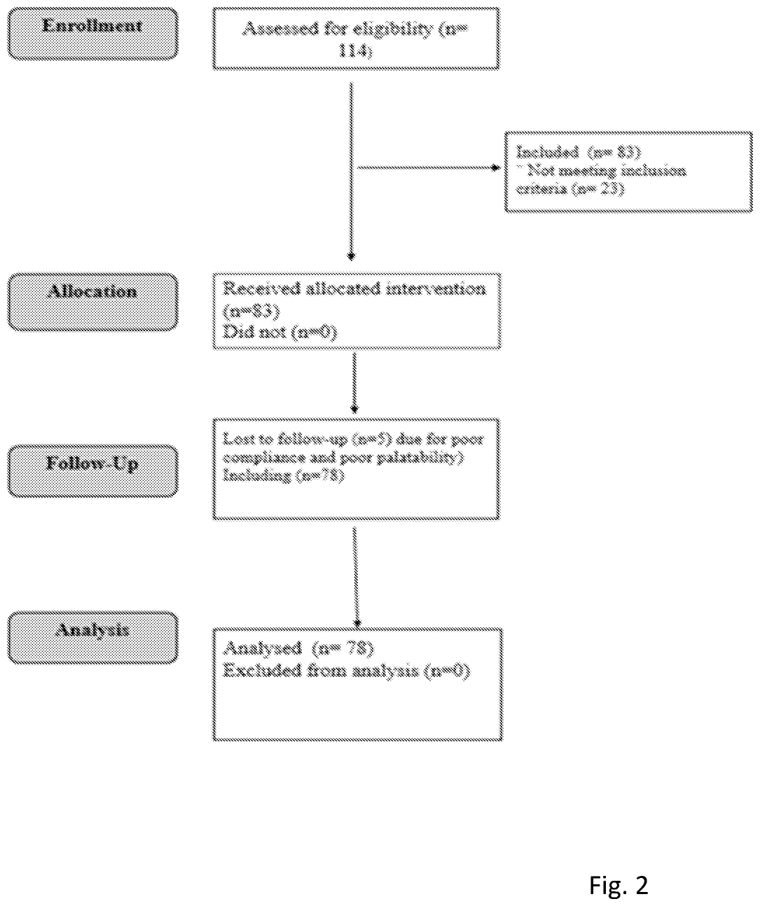

FIG. 2. Flow diagram of the survey among consumers of the food supplement aimed at relieving fatigue.

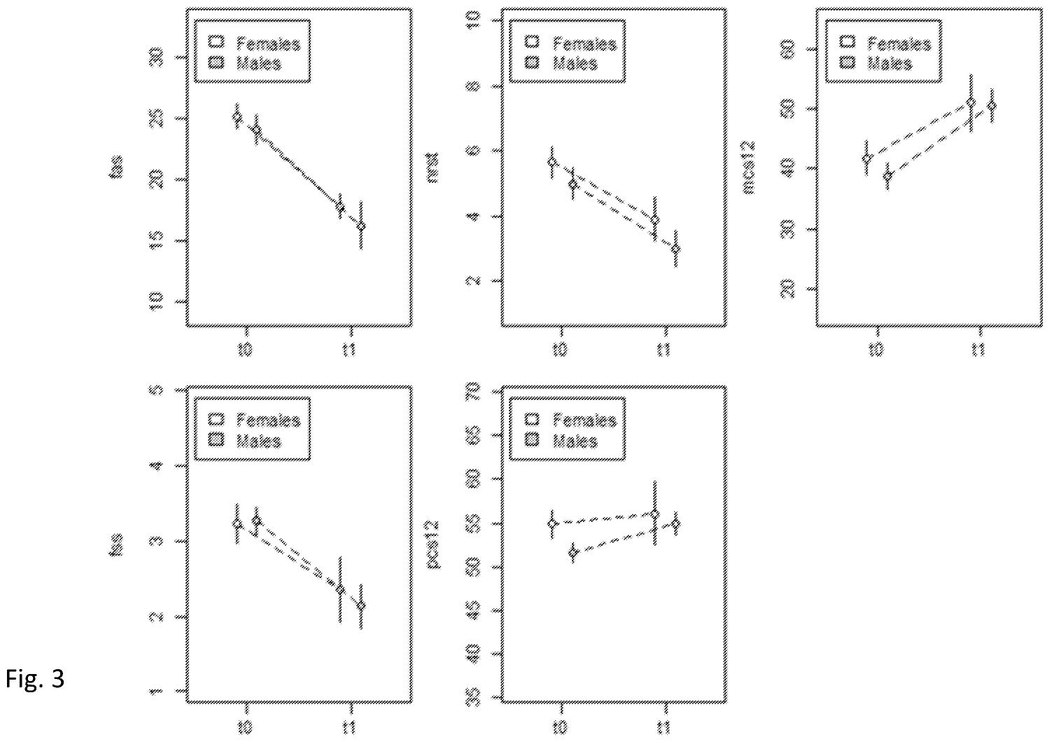

FIG. 3. Variation in the intensity (average and 95% confidence intervals) of the fatigue (FAS, FSS and NRST scores) and quality of the life (SF12 in the physical component, pcs12, and mental component mcs12) observed in the subjects recruited, at the beginning (t0) and at the end (t1) of the intake of the pomegranate extract, B vitamins and vitamin C-based food supplement.

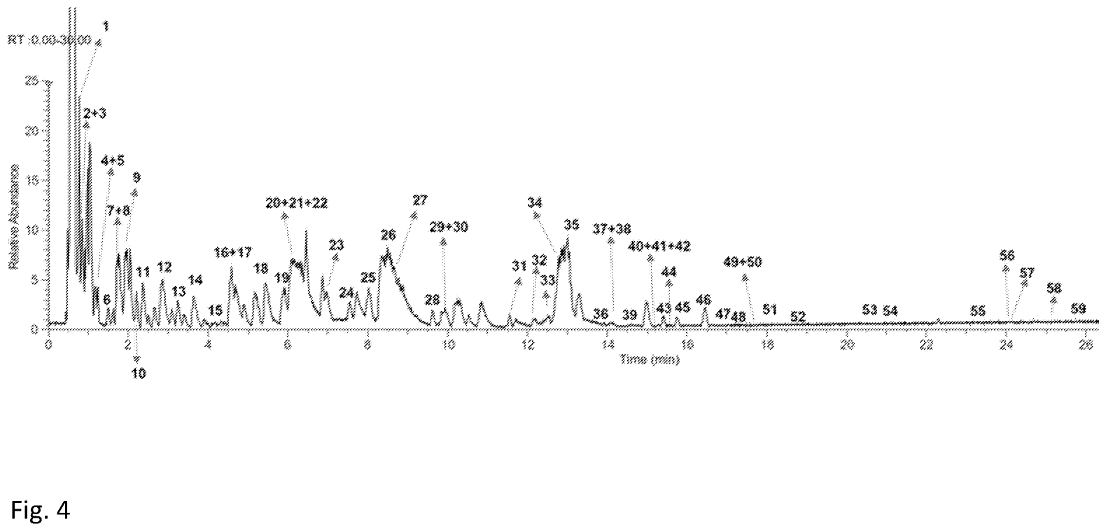

FIG. 4. The Reversed phase (RP) ultra-high-performance liquid chromatography (UHPLC) coupled to quadrupole-time-of-flight (QTOF) mass spectrometry in tandem analysis on the pure pomegranate liquid extract (without excipients).

### EXAMPLES

### Example 1

### In Vitro Antioxidant Activity Test (“DPPH Radical Scavenging”)

To test the in vitro antioxidant potential of the compositions of the invention, a DPPH (2,2-diphenyl-1-picrylhydrazyl) assay was carried out based on the measurement of the discoloration of the DPPH radical at 520 nm after reaction with the antioxidant components present in the extracts and compositions tested. The antioxidant capacity of the tested compounds is measured based on their DPPH reduction potential. The experiment was conducted according to the method described by Blois (Blois M S, 1958).

Ascorbic acid is used as a control.

The DPPH percentage of sample discoloration was calculated with the following formula:

% DPPH scavenging=[(Acontrol−Aextract/Acontrol)]×100, where A is absorbance.

| | | |
|---|---|---|
| TESTED COMPOSITION | concentration | % discoloration |

| | | | |
|---|---|---|---|
| Composition 1 (C1) | 5 | mg/ml | 99.2 ÷ 0.3 |
| Composition 2 (C2) | 5 | mg/ml | 98.1 ÷ 0.4 |
| Vitamin C | 0.11 | mg/ml | 91.7 ÷ 0.2 |
| Pomegranate titrated in | 5 | mg/ml | 70.3 ÷ 0.9 |
| polyphenols at 20%* | | | |
| Vitamin D | 5 | mg/ml | 55.1 ÷ 0.4 |
| B Group Vitamin complex | 5 | mg/ml | NS** |

*Dry extract
**non-significant result = 0

The individual extracts are the same as those used in the compositions.

### Composition 1:

Pomegranate extract (68% w/w of the composition), B group vitamin complex (5% w/w), vitamin C (27% w/w).

### Composition 2:

Pomegranate extract (99.99% w/w), vitamin D (0.01% w/w).

### Synergistic Effect:

The synergistic effect was evaluated according to the method described by S. R. Colby in “Calculation of the synergistic and antagonistic responses of herbicide combinations” Weeds, 1967.

The synergy factor was calculated for each compound. A factor of >1 indicates the existence of a synergistic effect. A factor of <1 indicates the existence of an antagonistic effect.

The formula applied in the calculations is as follows:

Expected rate of efficacy=A+B−(A*B/100)

Where the efficacy rate corresponds to the % of discoloration of the sample, i.e. the antioxidant capacity of the tested compound.

Synergy Factor (SF)=(1*Observed Rate of Efficacy (%))/Expected Rate of Efficacy (%)

Calculation of the synergy factor for A+B=C1 where A=pomegranate extract and B=vitamin C. C1=composition 1.

| | Observed rate of | | | Expected rate | |
|---|---|---|---|---|---|
| | efficacy (%) | | | of efficacy | Synergy |
| | A | B | C1 | for C1 | Factor |
| DPPH radical scavenging | 70.3 | 91.7 | 99.2 | 97.5% | 1.02 |

Calculation of the synergy factor for A+B=C2 where A=pomegranate extract, B=vitamin D, C2=composition 2.

| | Observed rate of | | | Expected rate | |
|---|---|---|---|---|---|
| | efficacy (%) | | | of efficacy | Synergy |
| | A | B | C2 | for C2 | Factor |
| DPPH radical scavenging | 70.3 | 55.1 | 98.1 | 86.7% | 1.13 |

The calculations show an SF>1 for all the compositions tested compared to the sum of the main ingredients with significant antioxidant activity (pomegranate+vitamin C, pomegranate+vitamin D).

The antioxidant effect shown for the compositions of the invention is capable of increasing the immune defences in patients to whom the composition is administered. In fact, free radical ROS have a negative action on the proper functioning of the immune system. Hence the antioxidant action of the present composition improves the immune defences of the patient to whom it is administered.

### Example 2

### Food Supplement

The food supplement contains vitamin C (ascorbic acid, 200 mg per 1 stick dose), and the following B vitamins: vitamin B₃ (niacin, 16 mg per 1 stick dose), vitamin B₂ (riboflavin, 7 mg per 1 stick dose), vitamin B₅ (pantothenic acid, 6 mg per 1 stick dose), vitamin B₁ (thiamine, 5.5 mg per 1 stick dose), vitamin B₆ (pyridoxine, 4 mg per 1 stick dose), vitamin B₉ (folic acid, 200 μg per 1 stick dose), vitamin B₈ (biotin, 50 μg per 1 stick dose), vitamin B₁₂ (cobalamin, 12.5 μg per 1 stick dose), and the whole fruit pomegranate dry extract (PunicaPLUS®, 500 mg per 1 stick dose). As far as PunicaPLUS® composition is concerned, it consists mainly of carbohydrates (95%) followed by fat (1%), protein (1%), and minerals (3%) with an energy value of 1711 KJ/100 g).

### Pomegranate Extract Analysis by UHPLC-HRMS

An aliquot of pomegranate dry extract (250 mg) was solubilized in methanol/water (1 mL, 30:70 v/v). The sample was then filtered through a cellulose acetate/cellulose nitrate mixed esters membrane (0.45 m; Millipore Corporation, Billerica, MA), and analyzed by RP-UHPLC-HRMS. UHPLC-HRMS analysis was performed on a Shimadzu Nexera UHPLC system, coupled online to a hybrid Ion Trap—Time of Flight Mass spectrometer (LCMS-IT-TOF, Shimadzu) equipped with an electrospray source (ESI).

For RP-UHPLC analysis a Kinetex Biphenyl 100 mm×2.1 mm, 2.6 μm (L×ID, particle size, Phenomenex®, Bologna, Italy) column was employed at a flow rate of 0.5 mL/min. The mobile phases consisted of A) 0.1% CH₃COOH in H₂O and B) CH₃CN plus 0.1% CH₃COOH. Analysis was performed to a gradient as follows: 0-25 min, 2-25% B; 25-28 min, 25-75% B; 28-31 min, 75-98% B. Column oven was set to 40° C., and a 2 μL sample was injected. PDA detection parameters were sampling rate 12 Hz, time constant 0.160 s and chromatograms were extracted at 280 and 330 nm. LC data elaboration was performed by LCMS Solution® software (Version 3.50.346, Shimadzu).

MS detection was performed in negative ESI ionization as follows: curve desolvation line (CDL), 250° C.; Block Heater, 250° C.; Nebulizing and Drying gas, 1.5 and 10 L/min; Capillary Voltage ESI(−): −3.5 kV; MS range, m/z 150-1500; ion accumulation time, 30 ms; ion trap repeat, 3. MS/MS was performed in a data dependent acquisition (DDA) mode, precursor ion selection was based on a base peak chromatogram (BPC) intensity of 500.000. Collision induced dissociation (CID), 50%, ion trap repeat. For analysis, the instrument was tuned using sodium trifluoroacetate (NaTFA). Metabolite annotation was based on accurate mass measurement, MS/MS fragmentation pattern, and comparison with in silico spectra from MS database searches (Mass bank of North America: https://mona.fiehnlab.ucdavis.edu/and Sirius: https://bio.informatik.uni-jena.de/software/sirius/). “Formula Predictor” software (Shimadzu) was used for the prediction of the molecular formula, using the following settings: maximum deviation from mass accuracy: 5 ppm, fragment ion information, and nitrogen rule.

### Evaluation of Efficacy and Tolerability

### Survey Population

One hundred and fourteen volunteers aged 18 to 75 with self-reported fatigue, who turned to their pharmacist requesting a food supplement for fatigue relief, were suggested to try a food supplement based on pomegranate extract, B vitamins, and vitamin C. The initial interview was conducted by the pharmacist. The involved subjects filled out the fatigue questionnaires (Fatigue Assessment Score—FAS, Fatigue Severity Score—FSS, 11-point Numeric Rating Scales—NRS), and the questionnaire on quality of life (Short Form 12 Health Survey—SF-12, subdivided into SF-12 mental and SF-12 physical), in the presence of the physician. The inclusion of the subjects was then evaluated by the physician, who assessed compliance with the inclusion and exclusion criteria. The criteria for inclusion were: the subject must have a sensation of mild to moderate fatigue and feeling of tiredness, the Fatigue Assessment Score (FAS) must be between 22 and 34, and the Fatigue Severity Score (FSS) must be lower than 5. As far as the exclusion criteria are concerned, subjects suffering from chronic diseases or comorbidities potentially explaining the fatigue, malabsorption, anorexia, psychiatric disorders, liver pathologies, heart disease, and kidney pathologies were excluded from this survey. Pregnant and lactating women were also excluded. Subjects with known hypersensitivity to one or more components of the food supplement or subjects using multivitamin supplements in the month before the survey were also excluded. All subjects received oral and written information concerning this survey before they gave their written consent to participate.

### Survey Design

A real-life survey was performed to evaluate the effectiveness of a food supplement based on pomegranate extract, vitamins B, and vitamin C, in healthy subjects as suggested by the pharmacist, through the evaluation of fatigue (FAS, FSS, and NRS), and quality of life (SF-12 mental and SF-12 physical). The assessment of the tolerability of the food supplement was performed through the evaluation of adverse reactions (ARs), using a form based on that used by the Italian Phytovigilance System (IPS) to report possible ARs after the ingestion of food supplements. Subjects who communicated symptoms related to mild to moderate fatigue to the pharmacist were directed to a separate room, where they received an exhaustive explanation of the survey by a pharmacist. The participants underwent two interviews: at recruitment (i.e. at the baseline, t0) and after 28 days (i.e. after a month of treatment, t1). At the recruitment, the inclusion of subjects to the survey was evaluated by the physician who validated their eligibility and health prior to participation. After they signed written consent, in addition to the self-completion of questionnaires administered by the physician (FAS, FSS and NRS, SF-12 mental and SF-12 physical), each subject was given a form to complete based on that used by the Italian Phytovigilance System (IPS), to report the possible ARs after the ingestion of food supplements. These forms were collected at the end of the survey (t1). All subjects took one stick pack per day containing 500 mg of pomegranate extract, 200 mg of ascorbic acid, 16 mg of niacin, 7 mg of riboflavin, 6 mg of pantothenic acid, 5.5 mg of thiamine, 4 mg of pyridoxine, 200 μg of folic acid, 50 μg of biotin, 12.5 μg of cobalamin, for one month.

After they signed the written consent, the first questionnaire submitted to the participants at t0 was the Fatigue Assessment Score (FAS), which is a 10-item questionnaire that explores how the subject usually feels. For each statement the subjects answers one of the five answers proposed (from “Never” to “Always”)[19]. The FAS evaluates overall fatigue. Five questions (1, 2, 4, 5, and 10) reflected physical fatigue and five questions (3 and 6-9) reflected mental fatigue. The total FAS score was calculated by summing the scores for all questions, with the total score ranging from 10 to 50. In short, the subjects were divided broadly into two categories based on FAS score: FAS score 10-21: no fatigue (normal), and FAS score 22-50: considerable fatigue. The considerable fatigue group was sub-grouped into mild—moderate fatigue (FAS score: 22-34) or severe fatigue (FAS score: 35-50)[20,21]. Minimal Important Difference (MID) from baseline to the end of the treatment was at least 4 points or 10% change in the patients' FAS score.

The second questionnaire submitted to the participants at t0 was the Fatigue Severity Score (FSS). It is a questionnaire of nine items, evaluating both physical and mental fatigue symptoms. Subjects were asked to choose a number from 1 to 7 based on their symptoms (1 indicated absolute disagreement with a statement, while 7 indicated full agreement) [22]. The answer to the questions referred to the previous two weeks. These two scales were chosen because they provided self-completion, and are easy to use and manage. In addition, FAS was recently used as a questionnaire for the evaluation of post-Covid-19 fatigue [23], and the FSS scale is the most common scale used, including for healthy subjects [24].

The third questionnaire submitted to the participants at t0 was the 11-point Numeric Rating Scale (NRS). Fatigue NRS is a scale used for the evaluation of fatigue in common clinical practice and clinical trials. It is a self-compilation, single-item, 11 point-horizontal scale from 0 to 10, where 0 means no fatigue and 10 means severe fatigue [25].

Finally, the fourth questionnaire submitted to the participants at t0 was SF-12. It was used to determine quality of life. The SF-12 questionnaire is a reduced version of the Short Form-36 Health Survey developed in the USA in the 1980s. SF-36 contains 36 questions in which the data are aggregated into 8 scales to investigate physical activity, role and physical health, physical pain, general health, vitality, social activities, role and emotional state, and mental health. SF-12 contains only 12 items from the SF-36 and it appears to provide a good reproduction of the SF-36, and is in use in many countries. SF-12 is subdivided into the evaluation of Physical Health (PCS) and Mental Health (MCS) and both have subgroups. For PCS there are two items on Physical Functioning (PF), two items on Role-Physical (RP), One Item about Bodily Pain (BP) and one about General Health (GH). For Mental Health (MCS), there is an item about Vitality (VT), one item about Social Functioning (SF), two items about Role-Emotional (RE) and two about Mental Health (MH) [26]. To calculate the final score, it is necessary to exclude values out of the range, and four of the items need inverting so that a higher score consistently indicates better health. The indicator variables are weighted using regression coefficients and are standardized by adding a constant (regression intercept) [27].

All the data collected by the physician was categorized into nine sections: the first section includes the data of the subject and informed consent (obligatory for all the subjects participating in the proposed survey); the second and third sections respectively concern the inclusion and exclusion criteria of the subjects; the fourth section summarizes any prior infections contracted within “the last period”, and how these (if any) may affect the current survey; in the fifth, sixth, seventh and eighth sections, FAS, FSS, NRS, and SF-12 scores were calculated for each subject, respectively. The last section concerns the final evaluation of the subject by the physician based on the data collected.

After 28 days of food supplement intake, the participants were contacted by the physician for the collection of the final data, subdivided into nine sections. The first section included the subject data. The second section concerned the compliance of participants. The third section investigated the presence of any adverse effects secondary to the intake of the food supplement (if any). The fourth section investigated the level of satisfaction of the food supplement by the subject. The fifth, sixth, seventh and eighth sections contained the FAS, FSS, SF-12, NRS scores, respectively. The last section concerned the final evaluation of the subject by the physician based on the data collected.

### Evaluated Variables

As far as sociodemographic characteristics are concerned, the age of the participants was considered to be a continuous variable, and the gender of the participants a two-levels factor. The primary endpoint was to investigate the efficacy of a 1 month-daily dose of food supplement to reduce mild/moderate fatigue and improve performance. This evaluation was measured at the beginning (t0) and at the end of the supplement food intake (t1) by the validated questionnaires FAS and FSS. The secondary outcomes considered were the assessment of the quality of life of the subjects through the administration of the quality of life questionnaire SF-12, at the baseline and at the end of the supplement food intake, NRS, and tolerability measured as the occurrence of AEs.

Therefore, the endpoints expected were the following: less perception of fatigue and improved quality of life after one month of food supplement intake, without any AEs.

### Statistical Analysis

The sample size calculation was made using a 1−β power value of 0.95 and a significance level α=0.05. The sample size was determined to be 84 participants, allowing for a 10% drop out rate. The objective of the statistical analysis was to compare the score values for the FAS, FSS, NRS, SF-12 (in the two physical and mental components) scales, measured at the beginning and at the end of the intake of the food supplement, and the characteristics of the subjects recruited (sex and age). The most suitable statistical analysis for this type of data is a linear mixed model with random intercept (LMM), in which the score value of the scales is the dependent variable, while the measurements (t0 and t1), the sex and age of the recruited subjects constitute the independent variables. The interaction sex×measurement has also been inserted to account for differential responses to measurements between men and women. The identity of the subjects was entered into the model as a random effect to control any differences due to the specific conditions of the persons recruited in the experiment.

### Results

### Pomegranate Extract Chromatographic Analysis

RP-UHPLC-UV-HRMS analysis of the whole fruit pomegranate dry extract (PunicaPLUS®) indicated the presence of 19 main compounds (FIG. 1) belonging to different classes such as ellagitannins, gallotannins, phenolic acids and flavonol glycosides (table 1). Among ellagitannins, peak 6 with molecular formula C₃₄H₂₂O₂₂ and fragments at 600 m/z deriving from loss of gallagic acid, was assigned as punicalin. Peak 9 and 15 with molecular formula C₃₄H₂₄O₂₂ and precursor ion at 783 m/z, showed fragments at m/z 765 and 481 m/z deriving from the loss of water and hexahydroxydiphenic acid respectively, leading to their tentative identification as pedunculagin. Peaks 10-12,13 were detected as doubly charged ions with molecular formula C₄₈H₂₈O₃₀ and fragments at 301 m/z and 601 m/z deriving from ellagic acid and gallagic acid losses. Ellagic acid and its glycosilated derivatives, peaks 16-18, were all characterized by the fragment at 301 m/z. Gallic acid (peak 3) derivatives, peaks 3-5, where characterized by the fragment at 169 m/z of gallic acid deriving from hexose moiety cleavage. Peak 19 with the molecular formula C20H16O12 showed a main fragment at 285 m/z, deriving from hexose loss, and was then assigned as Kaempferol hexoside. Peaks 7, 14, were characterized by fragments at 463, 301 and 169, deriving respectively from the losses of ellagic acid-hexose, ellagic and gallic acid losses and were tentatively assigned as HHDP galloyl hexose, while peak 11 with molecular formula C27H22O19 and similar fragmentation was hypothesized as a Galloyl-HHDP-gluconate derivate. Ellagitannins, including to different derivatives of galloyl (7.96%), hexahydroxydiphenoyl (HHDP) (12.28%), and punicalgin isomers (66%) were the most abundant compounds, followed by small amounts of organic acids, represented by citric (1.2%) and glycosylated flavonoids (1%).

TABLE 1

| | | | Mol. | [M − H]−/ | | Error |
|---|---|---|---|---|---|---|
| Peak | Tr | Compound | formula | [M − 2H]2− | [MS/MS] | (ppm) |
| 1 | 1.25 | HHDP-hexose | C20H18O14 | 481.0693 | 300, 275 | 6.9 |
| 2 | 1.51 | Citric acid | C6H8O7 | 191.0194 | 111, 173 | 0.85 |
| 3 | 1.6 | Galloyl-hexoside | C13H15O10 | 331.0577 | 211, 169 | 1.54 |
| 4 | 1.74 | Gallic acid | C7H6O5 | 169.0158 | 125, 107 | 0 |
| 5 | 2.6 | Galloyl-hexoside* | C13H15O10 | 331.0577 | 211, 169 | 1.54 |
| 6 | 2.85 | Punicalin β | C34H22O22 | 781.0593 | 721, 601, 575, | 0 |
| | | | | | 392, 298, 273, | |
| 7 | 3.3 | HHDP gallovl hexose | C27H22O18 | 633.0867 | 463, 300, 275, | −1.3 |
| | | | | | 249, 169, 125 | |
| 8 | 3.67 | Citric acid derivative | C17H12O11 | 391.0268 | 270 | −4.35 |
| 9 | 4.03 | Pedunculagin (di- | C34H24O22 | 783.0629 | 481, 300 275, | 0 |
| | | HHDP-hexose) | | | 249 | |
| 10 | 4.29 | Punicalagin* | C48H28O30 | 541.0266* | 301, 601, 275 | −0.27 |
| 11 | 4.76 | Galloyl-HHDP- | C27H22O19 | 649.0617 | 301, 497, 626 | −1.3 |
| | | gluconate | | | | |
| 12 | 6.35 | Punicalagin** | C48H28O30 | 541.0266 | 301, 601, 275 | −0.27 |
| 13 | 10.08 | Punicalagin** | C48H28O30 | 541.0266 | 301, 601, 275 | −0.27 |
| 14 | 14.49 | HHDP galloyl hexose* | C27H22O18 | 633.0867 | 463, 300, 275, | −1.3 |
| | | | | | 249, 169, 125 | |
| 15 | 15.54 | Pedunculagin (di- | C34H24O22 | 783.0629 | 481, 300 275, | 0 |
| | | HHDP-hexose)* | | | 249 | |
| 16 | 18.73 | Ellagic acid-hexoside | C26H32O13 | 551.1749 | 301, 389, 341 | −4.92 |
| 17 | 23 | Ellagic acid | C14H6O8 | 301.0029 | 229, 284, 267 | −4.35 |
| 18 | 23.81 | Ellagic acid-pentoside | C19H14O12 | 433.0413 | 301 | 0 |
| 19 | 24.26 | Kaempferol hexoside | C20H16O12 | 447.0167 | 285, 255 | 5.3 |

*[M − 2H]2−
**Isomer

Compounds identified in pomegranate dry extract (PunicaPLUS®) according to their retention time (RT), compound, molecular formula, m/z and MS/MS, mass accuracy (Δppm), reported as part per million error

### Survey Among Consumers of the Evaluation of Efficacy and Tolerability of the Food Supplement

The survey flow chart is reported in FIG. 2.

The participants had similar sociodemographic characteristics, with no significant difference from each other (Table 2).

TABLE 2
Demographic characteristics of the recruited subjects.
Demographic characteristics

| | |
|---|---|
| Man | 21 |
| Woman | 57 |
| Age in Men | 42.5 ± 14.5 |
| Age in Women | 39.8 ± 13.8 |

In Table 3, the descriptive values (mean, standard deviation and range of values) are given for the score of each assessment scale measured in men and women at t0 and t1.

TABLE 3
Observed values (mean, standard deviation, minimum and maximum)
for fatigue (FAS, FSS and NRS scales) and quality of life
(SF-12) observed in the involved subjects at the beginning
(t0) and at the end (t1) of the food supplement intake.

| | Woman | | Man | |
|---|---|---|---|---|
| Scale | t0 | t1 | t0 | t1 |
| FAS | 25.2 ± 3.7 | 17.8 ± 4.5 | 24.1 ± 2.2 | 16.2 ± 4.4 |
| | (10-33) | (11-29) | (22-30) | (9-24) |
| FSS | 3.2 ± 1 | 2.4 ± 0.7 | 3.3 ± 1.0 | 2.1 ± 0.7 |
| | (1.2-4.8) | (1.2-4.6) | (1.6-4.9) | (1-3.8) |
| NRS | 5.6 ± 1.8 | 3.9 ± 1.8 | 5.0 ± 1.5 | 3.0 ± 1.2 |
| | (1-10) | (1-8) | (2-8) | (2-6) |
| SF12 - physical | 54.9 ± 5.8 | 56.0 ± 4.3 | 51.6 ± 8.3 | 54.9 ± 2.9 |
| | (35.8-65.8) | (41.2-69.2) | (36-64.6) | (48.5-61.5) |
| SF12 - mental | 41.9 ± 10.6 | 51.1 ± 8.1 | 38.8 ± 11.2 | 50.7 ± 6.2 |
| | (15.7-58.8) | (19-64.8) | (19-58.7) | (35.3-58.7) |

### Primary Outcomes: FAS, FSS and NRST

The LMM model (Table 4) identified a statistically significant effect for measurements and an effect near to the significance threshold for sex, while no significant effects were identified for age or the interaction between measurements and sex. In detail, the value of the FAS score decreased between t0 and t1 (FIG. 3) in both men (−7.9±1.2, t76=6.665, P=0.001), and women (−7.4±0.7, t76=10.322, P=0.001). Men seemed to have lower values than women (t0: −1.04±1.01, t150=1.027, P=0.30; t1: −1.51±1.01, t150=1.492, P=0.13). The random effect between the subjects was not statistically significant (LRχ²=0.351, P=0.55).

The LMM model for the FSS scale (Table 4) provided results similar and consistent to those observed in the previous analysis for the FAS scale. The only statistically significant effect was the measurement effect, while no significant effects emerged for sex, age and the sex×measurement interaction. In detail, the FSS score significantly decreased from t0 to t1 (FIG. 3), in both men (−0.87±0.11, t76=7.737, P=0.001) and women (−1.14±0.18, t76=6.132, P=0.001). The random effect of the subject was statistically significant (LRχ²=22.075, P=0.001), suggesting that a portion of the variability in the FSS score was due to among-subjects variability not related to sex, age, and experimental measurements.

The LMM model (Table 4) for the NRS scale identified a statistically significant effect for both the measurement and sex, while no significant effect was identified for age or for the interaction between measurement and sex. In detail, the NRS score significantly decreased between t0 and t1 (FIG. 3) with a similar pattern in women (−1.73±0.31, t76=5.611, P=0.001) and men (−2.00±0.51, t76=3.922, P=0.001). Furthermore, here too, men showed lower NRS scores than women, although the difference was not statistically significant at measurement t0 (−0.67±0.43, t150=1.515, P=0.13), but it was at measurement t1 (−0.93±0.44, t150=2.115, P=0.036). The random effect between the subjects was not statistically significant (LRχ²=0.753, P=0.53).

### Secondary Endpoint: SF-12 Physical and SF-12 Mental

The LMM model (Table 4) for the physical component of the SF-12 scale identified a statistically significant effect for both measurement and sex, while no significant effect emerged for age or the interaction between measurement and sex. In detail, the value of the SF-12-physical index increased from measurement t0 to measurement t1, but the response was significant only for men (+3.34±1.59, t76=2.095, P=0.039, FIG. 3), while for women the variation was not significant (+1.19±0.97, t76=1.234, P=0.22, FIG. 3). Women showed higher values than men, although the difference was statistically significant only at measurement t0 (t0: +3.23±1.39, t150=2.321, P=0.022, t1: +1.08±1.39, t150=0.781, P=0.44; FIG. 3). Finally, the random effect between the subjects was not statistically significant (LRχ²=0.753, P=0.39). The LMM model (Table 4) for the mental component of the SF-12 scale identified a statistically significant effect for measurements only, while no significant effects emerged for sex, age or the interaction between measurement and sex. In detail, the value of the SF-12-mental index significantly increased from t0 to t1 (FIG. 3) in both women (+9.22±1.75, t151=5.261, P0.001) and men (+11.88±2.89, t151=17.588, P=0.001, FIG. 3). As in the previous analysis, the random effect was not statistically significant (LRχ²=0.001, P=0.99).

TABLE 4
Results of LMM models for FAS, FSS and NRS fatigue scales
and quality of life (physical and mental SF-12).

| Modello | F | gdl | P |
|---|---|---|---|
| FAS | | | |
| Measurement | 122.17 | 1.76 | <0.001 |
| Sex | 2.960 | 1.75 | 0.089 |
| Age | 1.433 | 1.75 | 0.230 |
| Measurement × sex | 0.117 | 1.76 | 0.730 |
| FSS | | | |
| Measurement | 85.694 | 1.76 | <0.001 |
| Sex | 0.134 | 1.75 | 0.710 |
| Age | 1.881 | 1.75 | 0.170 |
| Measurement × sex | 1.507 | 1.76 | 0.220 |
| NRS | | | |
| Measurement | 39.239 | 1.76 | <0.001 |
| Sex | 6.119 | 1.75 | 0.016 |
| Age | 0.331 | 1.75 | 0.570 |
| Measurement × sex | 0.195 | 1.76 | 0.660 |
| SF12 - physical component | | | |
| Measurement | 5.909 | 1.76 | 0.017 |
| Sex | 4.361 | 1.75 | 0.040 |
| Age | 0.029 | 1.75 | 0.860 |
| Measurement × sex | 1.323 | 1.76 | 0.250 |
| SF12 - mental component | | | |
| Measurement | 39.029 | 1.76 | <0.001 |
| Sex | 0.961 | 1.75 | 0.340 |
| Age | 0.188 | 1.75 | 0.670 |
| Measurement × sex | 0.620 | 1.76 | 0.430 |

### Tolerance and Safety Assessment

For the evaluation of tolerance of the pomegranate extract, B vitamins and vitamin C-based food supplement, adverse events were monitored throughout the whole period through spontaneous reporting of adverse events (AEs) by the participants to the physician. During the 1 month of food supplement intake, no subjects reported adverse effects (AEs) related to this food supplement, and the physician judged that the food supplement was considered well tolerated.

### Discussion

Vitamin deficiency and chronic inflammation seem to be possible causes of fatigue and growing evidence suggests that fatigue can be improved through a nutritional intervention [14] aimed at reducing marginal vitamin deficiencies and chronic inflammation. Thus, inventors investigated whether a food supplement consisting of the extract obtained from pomegranate, which shows anti-inflammatory activity, B vitamins and vitamin C can help fight SF.

The dry extract from the whole fruit of pomegranate, obtained through a hydroalcoholic extraction and used as an ingredient for a food supplement in a hydrosoluble powder dosage form, was analysed through a chromatographic method coupled with a hybrid Ion Trap—Time of Flight Mass spectrometer. The extract mainly consists of polyphenols and organic acids. The four main polyphenolic families are ellagitannins (punicalagin, ellagic acid hexose, ellagic acid-pentoside, punicalagin di HFHDP hexose, punicalin β), gallotannins (HIDP-hexose, galloyl-hexoside, galloyl-HIDP-gluconate, HHIDP galloyl hexose), phenolic acids (ellagic acid and gallic acid) and flavonol glycosides (kaempferol hexoside). The identified compounds are in line with literature data as pomegranate extract is rich in polyphenolic compounds, flavonoids and phenolic acids, as well as tannins, which are mainly represented by ellagitannins. In agreement with literature data, the main ellagitannins found in the analysed pomegranate extract are punicalagin and punicalin, which are the prominent polyphenols isolated from pomegranate fruit [28]. Moreover, citric acid is the most represented organic acid in the pomegranate extract.

After the chemical characterization of the pomegranate extract, a survey among consumers of dietary supplements aimed at alleviating fatigue was conducted to obtain preliminary results on the efficacy and tolerability on prolonged fatigue of a food supplement based on a combination of the chemically characterized pomegranate extract, B vitamins, and vitamin C, in healthy consumers for an intake period of one month.

The method used to perform this survey consists of: 1) power analysis calculation, to determine the number of subjects involved in the survey, 2) definition of inclusion and exclusion criteria, to accomplish the purpose of the survey, 3) the use of fatigue and quality of life questionnaires validated in the general population available in the Italian language, and 4) the use of inferential statistics to analyze the obtained results.

The results of the survey clearly show that the daily intake of the food supplement based on pomegranate extract, B vitamins (at doses ranging from 50 to 500% of the daily reference intake for vitamins in adults), and vitamin C (at a dose of 250% of the daily reference intake for vitamin C), for a period of one month may yield a significant improvement in fatigue and quality of life. In fact, after supplementation, FAS, FSS, and NRT scores significantly decreased between t0 and t1 and, the quality of life was consequently improved, as both SF-12 scores (in the two physical and mental components) significantly increased. In addition, the food supplement was considered well tolerated by the physician as no subjects reported adverse effects (AEs) related to consumption of this food supplement.

In conclusion, this survey of consumers of dietary supplements aimed at alleviating fatigue provides data on the effects on prolonged fatigue and the tolerability of a food supplement based on the combination of chemically characterized pomegranate extract, B vitamins, and vitamin C, in healthy consumers for one month of intake.

### Example 3

### RP-UHPLC-ESI-MS/MS Punica PLUS™

The Reversed phase (RP) ultra-high-performance liquid chromatography (UHPLC) coupled to quadrupole-time-of-flight (QTOF) mass spectrometry in tandem analysis on the pure pomegranate liquid extract (without excipients) showed the presence of 59 molecules, including punicalagin, ellagic acid and gallic acid.

The most abundant molecules are citric acid (peaks 1), Punicalagins (peaks 17, 20, 26), ellagic acid (peaks 35), followed by gallic acid and derivatives (FIG. 4).

TABLE 5

| | | | Mol. | [M − H]−/ | | Error | |
|---|---|---|---|---|---|---|---|
| Peak | Tr | Compound | formula | [M − 2H]2− | [MS/MS] | (ppm) | Intensity |
| 1 | 0.65 | Citric acid | C6H8O7 | 191.0194 | 111, 173 | 2.01 | 2.08E+11 |
| 2 | 0.92 | Galloyl-hexoside | C13H15O10 | 331.0577 | 211, 169 | 1.54 | 1.23E+08 |
| 3 | 0.99 | HHDP-hexose | C20H18O14 | 481.0693 | 300, 275 | 6.9 | 1.28E+09 |
| 4 | 1.08 | Gallic acid | C7H6O5 | 169.0133 | 125 | 0.99 | 2.60E+10 |
| 5 | 1.16 | Galloyl-hexoside_I | C13H15O10 | 331.0577 | 211, 169 | 1.52 | 2.29E+07 |
| 6 | 1.44 | HHDP galloyl | C27H22O18 | 633.0739 | 275, 300 | 0.49 | 3.78E+08 |
| | | hexose | | | | | |
| 7 | 1.77 | Punicalin β | C34H22O22 | 781.0593 | 721, 601, | 0.1 | 6.04E+08 |
| | | | | | 575, 392, | | |
| | | | | | 298, 273, | | |
| 8 | 1.87 | Protocatecuic acid | C7H6O4 | 153.0184 | 109 | 0.97 | 5.50E+08 |
| 9 | 1.98 | Punicalin β_I | C34H22O22 | 781.0593 | 721, 601, | 0.11 | 6.27E+08 |
| | | | | | 575, 392, | | |
| | | | | | 298, 273, | | |
| 10 | 2.38 | HHDP galloyl | C27H22O18 | 633.0739 | 275, 300 | 0.5 | 7.92E+09 |
| | | hexose_I | | | | | |
| 11 | 2.52 | Epigallocatechin | C15H14O7 | 305.0673 | 261, 219, | 0.58 | 1.54E+08 |
| | | | | | 179 | | |
| 12 | 2.84 | Pedunculagin (di- | C34H24O22 | 783.0629 | 481, 300 | 2.75 | 2.77E+08 |
| | | HHDP-hexose) | | | 275, 249 | | |
| 13 | 3.24 | HHDP galloyl | C27H22O18 | 633.0740 | 275, 300 | 0.47 | 5.38E+09 |
| | | hexose_II | | | | | |
| 14 | 3.69 | Punicalagin | C48H28O30 | 541.0262** | 301, 601, | 2.49 | 8.36E+07 |
| | | | | | 275 | | |
| 15 | 4.16 | HHDP galloyl | C27H22O18 | 633.0738 | 275, 300 | 0.51 | 1.92E+08 |
| | | hexose_III | | | | | |
| 16 | 4.57 | Hamamelitannin | C20H20O14 | 483.0773 | 169, 271, | 0.76 | 5.17E+08 |
| | | | | | 313 | | |
| 17 | 4.59 | Punicalagin_I | C48H28O30 | 541.0262** | 301, 601, | 2.49 | 1.85E+08 |
| | | | | | 275 | | |
| 18 | 5.44 | Pedunculagin (di- | C34H24O22 | 783.0629 | 481, 300 | 2.75 | 2.77E+08 |
| | | HHDP-hexose)_I | | | 275, 249 | | |
| 19 | 5.98 | HHDP galloyl | C27H22O18 | 633.0740 | 275, 300 | 0.47 | 2.22E+09 |
| | | hexose_IV | | | | | |
| 20 | 6.10 | Punicalagin_II | C48H28O30 | 541.0266** | 301, 601, | 2.49 | 2.44E+08 |
| | | | | | 275 | | |
| 21 | 6.12 | 2-hydroxycinnamic | C9H8O3 | 163.0393 | 119 | 0.79 | 6.10E+07 |
| | | acid | | | | | |
| 22 | 6.15 | p-Coumaric acid | C15H18O8 | 325.0929 | 163, 119 | 0.12 | 317874400 |
| | | hexoside | | | | | |
| 23 | 6.96 | Pedunculagin (di- | C34H24O22 | 783.0629 | 481, 300 | 2.75 | 2.27E+08 |
| | | HHDP-hexose)_II | | | 275, 249 | | |
| 24 | 7.92 | 1,3,6-tri-O- | C27H24O18 | 635.0881 | 483, 465, | 3.65 | 1.42E+07 |
| | | galloylhexose | | | 169 | | |
| 25 | 8.14 | Eriodictyol-7-O- | C21H22O11 | 449.1094 | 287, 259 | 2.47 | 2.99E+08 |
| | | hexoside | | | | | |
| 26 | 8.47 | Punicalagin | C48H28O30 | 541.0266** | 301, 601, | 2.49 | 2.83E+08 |
| | | | | | 275 | | |
| 27 | 8.77 | HHDP galloyl | C27H22O18 | 633.0740 | 275, 300 | 0.47 | 4.13E+08 |
| | | hexose_V | | | | | |
| 28 | 9.25 | 1,3,6-tri-O- | C27H24O18 | 635.0881 | 483, 465, | 3.65 | 1.65E+07 |
| | | galloylhexose_I | | | 169 | | |
| 29 | 9.62 | 1,3,6-tri-O- | C27H24O18 | 635.0880 | 483, 465, | 3.63 | 6.02E+06 |
| | | galloylhexose_II | | | 169 | | |
| 30 | 9.93 | 1,3,6-tri-O- | C27H24O18 | 635.0882 | 483, 465, | 3.61 | 5.35E+06 |
| | | galloylhexose_III | | | 169 | | |
| 31 | 11.22 | 1,3,6-tri-O- | C27H24O18 | 635.0883 | 483, 465, | 3.61 | 1.11E+07 |
| | | galloylhexose_IV | | | 169 | | |
| 32 | 11.56 | Myricetin 3-O-beta- | C21H20O13 | 479.0844 | 316 | 2.58 | 2.05E+07 |
| | | L-galactopyranoside | | | | | |
| 33 | 11.72 | 1,3,6-tri-O- | C27H24O18 | 635.0883 | 483, 465, | 3.61 | 2.19E+07 |
| | | galloylhexose_V | | | 169 | | |
| 34 | 12.73 | quercetin 3-O- | C21H18O13 | 477.0669 | 301 | 1.17 | 1.17E+07 |
| | | glucuronide | | | | | |
| 35 | 13.01 | Ellagic Acid | C14H6O8 | 300.9987 | 229 | 3.75 | 6.98E+08 |
| 36 | 13.54 | Quercetin-3-O- | C27H30O16 | 609.1458 | 301, 463 | 3.52 | 4.34E+07 |
| | | Rutinoside | | | | | |
| 37 | 13.89 | Naringenin-7-O- | C21H22O10 | 433.1143 | 271, 313 | 3.36 | 3.71E+06 |
| | | hexoside | | | | | |
| 38 | 13.91 | quercetin 3-O- | C21H20O12 | 463.0895 | 301 | 4.54 | 5.22E+07 |
| | | hexoside | | | | | |
| 39 | 14.59 | quercetin 3-O- | C21H18O13 | 477.0669 | 301 | 1.17 | 1.08E+07 |
| | | glucuronide | | | | | |
| 40 | 15.24 | quercetin-3- | C20H18O11 | 433.0780 | 301 | 3.95 | 5.47E+07 |
| | | Arabinoside | | | | | |
| 41 | 15.27 | Eryodictol | C15H12O6 | 287.0568 | 259, 125 | 4.39 | 3.20+E06 |
| 42 | 15.38 | kaempferol 7-O- | C21H20O11 | 447.0943 | 285, 299 | 3.56 | 1.59E+07 |
| | | hexoside | | | | | |
| 43 | 15.40 | Kaempferol-3-O- | C27H30O15 | 593.1523 | 285 | 3.27 | 1.09E+08 |
| | | glucorhamnoside | | | | | |
| 44 | 15.62 | quercetin-3- | C20H18O11 | 433.0780 | 301 | 3.95 | 3.86E+05 |
| | | Arabinoside_I | | | | | |
| 45 | 15.75 | kaempferol 7-O- | C21H20O11 | 447.0943 | 285, 299 | 3.56 | 9.14E+07 |
| | | hexoside_I | | | | | |
| 46 | 16.41 | 1,2,3,6- | C34H28O22 | 787.1018 | 617, 465, | 1.7 | 4.69E+05 |
| | | tetragalloylglucose | | | 169 | | |
| 47 | 16.63 | myricetin | C15H10O8 | 317.0309 | 178, 151 | 3.50 | 3.17E+06 |
| 48 | 16.99 | Kaempferol 3- | C20H18O10 | 417.0833 | 284 | 2.88 | 1.11E+07 |
| | | alpha-L- | | | | | |
| | | arabinopyranoside | | | | | |
| 49 | 17.31 | Apigenin 8-C- | C21H20O10 | 431.0996 | 269 | 3.58 | 3.52E+07 |
| | | hexoside | | | | | |
| 50 | 17.35 | Kaempferol 3- | C20H18O10 | 417.0833 | 284 | 2.88 | 9.85E+06 |
| | | alpha-L- | | | | | |
| | | arabinopyranoside_I | | | | | |
| 51 | 17.57 | Phloridzin | C21H24O10 | 435.1304 | 273, 167 | 3.64 | 1.67E+07 |
| 52 | 19.11 | Hesperidin | C28H34O15 | 609.1816 | 301 | 3.11 | 5.37E+06 |
| 53 | 20.50 | Quercetin | C15H10O7 | 301.0359 | 178, 151 | 1.29 | 1.03E+07 |
| 54 | 20.90 | Kaempferol | C15H10O6 | 285.0402 | 151, 133 | 4.72 | 5.31E+06 |
| 55 | 23.58 | Naringenin | C15H12O5 | 271.0613 | 177, 151, | 5.01 | 1.38E+06 |
| | | | | | 119 | | |
| 56 | 23.99 | Apigenin | C15H10O5 | 269.0453 | 187, 119 | 3.5 | 1.65E+06 |
| 57 | 24.06 | Luteolin | C15H10O6 | 285.0402 | 133, 151 | 4.83 | 5.57E+06 |
| 58 | 25.48 | Isorhamnetin | C16H12O7 | 315.0517 | 300 | 4.25 | 1.48E+06 |
| 59 | 25.80 | Syringetin | C17H14O8 | 345.0612 | 315, 330 | 3.87 | 4.72E+06 |

I = isomer
**doubly charged

### Method

Analyses were performed on a Thermo Ultimate RS 3000 coupled online to a Q-Exactive hybrid quadrupole Orbitrap mass spectrometer (Thermo Fisher Scientific, Bremen, Germany) equipped with a heated electrospray ionization probe (HESI II). For RP-UHPLC analysis a Kinetex Biphenyl 100 mm×2.1 mm, 2.6 μm (L×ID, particle size, Phenomenex®, Bologna, Italy) column was employed at a flow rate of 0.4 mL/min. The mobile phases consisted of A) 0.1% CH3COOH in H2O and B) ACN plus 0.1% CH3COOH. Analysis was performed in gradient as follows: 0-30.0 min, 2-30% B; 30-38 min, 30-98% B; 99% B hold for 2 min; returning to initial conditions in 0.1 min. Column oven was set to 40° C., 5 μL sample was injected. HRMS analysis was performed with Full MS (m/z 100-850) and data-dependent acquisition (dd-MS2 topN=5). A resolution of 35.000 and 15,000 FWHM at m/z 200 was selected respectively. Stepped normalized collision energy (NCE) with values of 15, 25 and 30 was used. Negative ESI− was employed. Source parameters were: Sheath gas pressure, 50 arbitrary units; auxiliary gas flow, 13 arbitrary units; spray voltage, −2.50 kV; capillary temperature, 260° C.; auxiliary gas heater temperature, 300° C., S-lens RF value: 30 arbitrary units. Metabolite annotation was performed by comparison with in silico MS/MS Natural Product Library of MS DIAL v 4.80 as previously reported (Sommella, E., Pagano, F., Salviati, E., Chieppa, M., Bertamino, A., Manfra, M., Sala, M., Novellino, E., & Campiglia, P. (2017). Chemical profiling of bioactive constituents in hop cones and pellets extracts by online comprehensive two-dimensional liquid chromatography with tandem mass spectrometry and direct infusion Fourier transform ion cyclotron resonance mass spectrometry. Journal of Separation Science).

### Examples of Compositions

### Composition 1

### Stick Pack to be Dissolved in Water

The composition of the present example is in the form of stick packs to be dissolved in water and comprises the following ingredients, as a percentage referred to the total weight of the composition: pomegranate 31.25%, vitamin C 12.5%, complex of vitamins of the B group 2.44% in addition to excipients including maltodextrins, silicon dioxide, steviol glycosides, beet red.

| | | Nutritional reference |
|---|---|---|
| List of ingredients | Mg per 1 stick | values for 1 stick |
| Pomegranate dry extract titrated at | 500 | — |
| 20% in polyphenols | | |
| Vitamin C | 200 | 250% |
| Niacin | 16 | 100% |
| Vitamin B2 | 7 | 500% |
| Pantothenic acid | 6 | 100% |
| Vitamin B1 | 5.5 | 500% |
| Vitamin B6 | 4 | 285.7% |
| Folate | 0.2 | 100% |
| Biotin | 0.05 | 100% |
| Vitamin B12 | 0.0125 | 500% |

Composition 1-bis:

### Tablets to be Swallowed

The composition of the present example is in the form of swallowable tablets. It includes the following ingredients, as a percentage referred the total weight of the composition: pomegranate 250%, vitamin C 800, complex of vitamins of the B group 400. The composition also comprises zinc, copper and iron. In addition, the composition comprises excipients such as cellulose, dicalcium phosphate as bulking agents, magnesium salts of fatty acids and silicon dioxide as anti-caking agents.

| | | Nutritional reference |
|---|---|---|
| List of ingredients | Mg per 1 tablet | values for 1 tablet |
| Pomegranate dry extract titrated at | 250 | — |
| 20 in polyphenols % | | |
| Vitamin C | 80 | 100% |
| Niacin | 16 | 100% |
| Vitamin B2 | 7 | 500% |
| Pantothenic acid | 6 | 100% |
| Vitamin B1 | 5.5 | 500% |
| Vitamin B6 | 4 | 285.7% |
| Folate | 0.2 | 100% |
| Biotin | 0.05 | 100% |
| Vitamin B12 | 0.0125 | 500% |
| Copper | 2 | 200% |
| Zinc | 10 | 100% |
| Iron | 30 | 214.3% |

### Composition 2:

### Liquid Solution in a Vial to be Drunk

The composition of the present example is in the form of a liquid solution in a vial to be drunk. It includes the following ingredients, as a percentage of the total weight of the composition: pomegranate 600, vitamin D 0.0005%. The composition comprises excipients such as water, xanthan gum as a thickener, sodium benzoate and potassium sorbate as preservatives, and steviol glycosides as a sweetener.

| | | Nutritional reference |
|---|---|---|
| List of ingredients | Mg for 1 vial | values for 1 vial |
| Pomegranate dry extract titrated | 300 | — |
| at 20% in polyphenols | | |
| Vitamin D | 0.025 | 500% |

Composition 2-bis:

### Sachet to be Dissolved in Water

The composition of the present example is in the form of powder in a sachet to be dissolved in water. It comprises the following ingredients, as a percentage of the total weight of the composition: pomegranate 20%, vitamin D 0.001%, vitamin C 2.6%. The composition also comprises excipients such as: maltodextrins, silicon dioxide as an anti-caking agent, steviol glycosides as a sweetener.

| | Mg per 1 | Nutritional reference |
|---|---|---|
| List of ingredients | sachet | values for 1 sachet |
| Pomegranate dry extract titrated | 600 | — |
| at 20% in polyphenols | | |
| Vitamin D | 0.050 | 1000% |
| Vitamin C | 80 | 100% |

### Composition 3:

### Chewable Tablet

| INGREDIENT | mg/tablet |
|---|---|
| VITAMIN D3 | 50 mcg |
| Pomegranate dry extract | 150 mg |
| D-Mannitol | |
| SUCRALOSE | |
| SORBITOL | |
| MAGNESIUM SALTS OF FATTY ACIDS | |
| SILICON DIOXIDE | |
| HYDROXYPROPYLCELLULOSE | |
| POMEGRANATE FLAVOR | |
| CITRIC ACID | |
| Steviol glycosides from STEVIA rebaudiana | |
| TOTALE | 550 mg |

### Composition 4:

### Sachet to be Dissolved in Water

| INGREDIENT | mg/sachet | |
|---|---|---|
| VITAMIN D3 | 50 | mcg |
| Pomegranate dry extract | 350 | mg |
| maltodextrines | | |
| SILICON DIOXIDE | | |
| POMEGRANATE FLAVOR | | |
| CITRIC ACID | | |
| Steviol glycosides from STEVIA rebaudiana | | |
| TOTALE | 3000 | mg |

### REFERENCES

1. Fukuda, K.; Dobbins, J. G.; Wilson, L. J.; Dunn, R. A.; Wilcox, K.; Smallwood, D. An epidemiologic study of fatigue with relevance for the chronic fatigue syndrome. J Psychiatr Res 1997, 31, 19-29, doi:10.1016/s0022-3956(96)00046-5.
2. Baek, Y.; Kim, H.; Mun, S.; Lee, S. Three-Component Herbal Tea Alleviates Prolonged Fatigue and Improves Sleep Quality: A Randomized Controlled Pilot Study. Explore (NY) 2018, 14, 420-423, doi:10.1016/j.explore.2018.05.001.
3. Zielinski, M. R.; Systrom, D. M.; Rose, N. R. Fatigue, Sleep, and Autoimmune and Related Disorders. Front Immunol 2019, 10, 1827, doi:10.3389/fimmu.2019.01827.
4. Jason, L. A.; Evans, M.; Brown, M.; Porter, N. What is fatigue? Pathological and nonpathological fatigue. PM R 2010, 2, 327-331, doi:10.1016/j.pmrj.2010.03.028.
5. Klimas, N. G.; Broderick, G.; Fletcher, M. A. Biomarkers for chronic fatigue. Brain Behav Immun 2012, 26, 1202-1210, doi:10.1016/j.bbi.2012.06.006.
6. Tardy, A. L.; Pouteau, E.; Marquez, D.; Yilmaz, C.; Scholey, A. Vitamins and Minerals for Energy, Fatigue and Cognition: A Narrative Review of the Biochemical and Clinical Evidence. Nutrients 2020, 12, doi:10.3390/nu12010228.
7. Portero, P.; Gomez—Merino, D. Stanchezza e motilita; EMC—Medicina Riabilitativa: 2013.
8. Yancey, J. R.; Thomas, S. M. Chronic fatigue syndrome: diagnosis and treatment. Am Fam Physician 2012, 86, 741-746.
9. Bucher, E.; Sandbakk, O.; Donath, L.; Roth, R.; Zahner, L.; Faude, O. Exercise-induced trunk fatigue decreases double poling performance in well-trained cross-country skiers. Eur J Appl Physiol 2018, 118, 2077-2087, doi:10.1007/s00421-018-3938-4.
10. Lin, C. F.; Lee, W. C.; Chen, Y. A.; Hsue, B. J. Fatigue-Induced Changes in Movement Pattern and Muscle Activity During Ballet Releve on Demi-Pointe. J Appl Biomech 2016, 32, 350-358, doi:10.1123/jab.2014-0263.
11. Le Mansec, Y.; Pageaux, B.; Nordez, A.; Dorel, S.; Jubeau, M. Mental fatigue alters the speed and the accuracy of the ball in table tennis. J Sports Sci 2018, 36, 2751-2759, doi:10.1080/02640414.2017.1418647.
12. Quera Salva, M. A.; Barbot, F.; Hartley, S.; Sauvagnac, R.; Vaugier, I.; Lofaso, F.; Philip, P. Sleep disorders, sleepiness, and near-miss accidents among long-distance highway drivers in the summertime. Sleep Med 2014, 15, 23-26, doi:10.1016/j.sleep.2013.06.018.
13. Nourbakhsh, B.; Revirajan, N.; Morris, B.; Cordano, C.; Creasman, J.; Manguinao, M.; Krysko, K.; Rutatangwa, A.; Auvray, C.; Aljarallah, S.; et al. Safety and efficacy of amantadine, modafinil, and methylphenidate for fatigue in multiple sclerosis: a randomised, placebo-controlled, crossover, double-blind trial. Lancet Neurol 2021, 20, 38-48, doi:10.1016/S1474-4422(20)30354-9.
14. Werbach, M. R. Nutritional strategies for treating chronic fatigue syndrome. Altern Med Rev 2000, 5, 93-108.
15. Haβ, U.; Herpich, C.; Norman, K. Anti-Inflammatory Diets and Fatigue. Nutrients 2019, 11, doi:10.3390/nu11102315.
16. Wang, P.; Zhang, Q.; Hou, H.; Liu, Z.; Wang, L.; Rasekhmagham, R.; Kord-Varkaneh, H.; Santos, H. O.; Yao, G. The effects of pomegranate supplementation on biomarkers of inflammation and endothelial dysfunction: A meta-analysis and systematic review. Complement Ther Med 2020, 49, 102358, doi:10.1016/j.ctim.2020.102358.
17. Ammar, A.; Bailey, S. J.; Chtourou, H.; Trabelsi, K.; Turki, M.; Hokelmann, A.; Souissi, N. Effects of pomegranate supplementation on exercise performance and post-exercise recovery in healthy adults: a systematic review. Br J Nutr 2018, 120, 1201-1216, doi:10.1017/S0007114518002696.
18. Urbaniak, A.; Skarpanska-Stejnborn, A. Effect of pomegranate fruit supplementation on performance and various markers in athletes and active subjects: A systematic review. Int J Vitam Nutr Res 2021, 91, 547-561, doi:10.1024/0300-9831/a000601.
19. Drent, M.; Lower, E. E.; De Vries, J. Sarcoidosis-associated fatigue. Eur Respir J 2012, 40, 255-263, doi:10.1183/09031936.00002512.
20. de Kleijn, W. P.; De Vries, J.; Wijnen, P. A.; Drent, M. Minimal (clinically) important differences for the Fatigue Assessment Scale in sarcoidosis. Respir Med 2011, 105, 1388-1395, doi:10.1016/j.rmed.2011.05.004.
21. De Vries, J.; Michielsen, H.; Van Heck, G. L.; Drent, M. Measuring fatigue in sarcoidosis: the Fatigue Assessment Scale (FAS). Br J Health Psychol 2004, 9, 279-291, doi:10.1348/1359107041557048.
22. Krupp, L. B.; LaRocca, N. G.; Muir-Nash, J.; Steinberg, A. D. The fatigue severity scale. Application to patients with multiple sclerosis and systemic lupus erythematosus. Arch Neurol 1989, 46, 1121-1123, doi:10.1001/archneur.1989.00520460115022.
23. Morgul, E.; Bener, A.; Atak, M.; Akyel, S.; Aktas, S.; Bhugra, D.; Ventriglio, A.; Jordan, T. R. COVID-19 pandemic and psychological fatigue in Turkey. Int J Soc Psychiatry 2021, 67, 128-135, doi:10.1177/0020764020941889.
24. Hjollund, N. H.; Andersen, J. H.; Bech, P. Assessment of fatigue in chronic disease: a bibliographic study of fatigue measurement scales. Health Qual Life Outcomes 2007, 5, 12, doi:10.1186/1477-7525-5-12.
25. Gladman, D.; Nash, P.; Goto, H.; Birt, J. A.; Lin, C. Y.; Orbai, A. M.; Kvien, T. K. Fatigue numeric rating scale validity, discrimination and responder definition in patients with psoriatic arthritis. RMD Open 2020, 6, doi:10.1136/rmdopen-2019-000928.
26. Gandek, B.; Ware, J. E.; Aaronson, N. K.; Apolone, G.; Bjorner, J. B.; Brazier, J. E.; Bullinger, M.; Kaasa, S.; Leplege, A.; Prieto, L.; et al. Cross-validation of item selection and scoring for the SF-12 Health Survey in nine countries: results from the IQOLA Project. International Quality of Life Assessment. J Clin Epidemiol 1998, 51, 1171-1178, doi:10.1016/s0895-4356(98)00109-7.
27. Ware, J. E.; Keller, S. D. SF-12: how to score the SF-12 physical and mental health summary scales; 2nd, Ed.; Boston MA: Health Institute, New England Medical Center: 1995.
28. Moga, M. A.; Dimienescu, O. G.; Balan, A.; Dima, L.; Toma, S. I.; Bigiu, N. F.; Blidaru, A. Pharmacological and Therapeutic Properties of Punica granatum Phytochemicals: Possible Roles in Breast Cancer. Molecules 2021, 26, doi:10.3390/molecules26041054.
29. Council, E. Commission Regulation (EU) No. 432/2012 establishing a list of permitted health claims made on foods, other than those referring to the reduction of disease risk and to children's development and health. 2012.
30. Council, E. Regulation (EC) No. 1924/2006 of the European Parliament and of the Council on nutrition and health claims made on foods. 2006.
31. Fahmy, H.; Hegazi, N.; El-Shamy, S.; Farag, M. A. Pomegranate juice as a functional food: a comprehensive review of its polyphenols, therapeutic merits, and recent patents. Food Funct 2020, 11, 5768-5781, doi:10.1039/d0fo01251c.
32. Lu, X. Y.; Han, B.; Deng, X.; Deng, S. Y.; Zhang, Y. Y.; Shen, P. X.; Hui, T.; Chen, R. H.; Li, X.; Zhang, Y. Pomegranate peel extract ameliorates the severity of experimental autoimmune encephalomyelitis via modulation of gut microbiota. Gut Microbes 2020, 12, 1857515, doi:10.1080/19490976.2020.1857515.
33. Trexler, E. T.; Smith-Ryan, A. E.; Melvin, M. N.; Roelofs, E. J.; Wingfield, H. L. Effects of pomegranate extract on blood flow and running time to exhaustion. Appl Physiol Nutr Metab 2014, 39, 1038-1042, doi:10.1139/apnm-2014-0137.
34. Torregrosa-Garcia, A.; Avila-Gandia, V.; Luque-Rubia, A. J.; Abellan-Ruiz, M. S.; Querol-Calderon, M.; Lopez-Roman, F. J. Pomegranate Extract Improves Maximal Performance of Trained Cyclists after an Exhausting Endurance Trial: A Randomised Controlled Trial. Nutrients 2019, 11, doi:10.3390/nu11040721.
35. Roelofs, E. J.; Smith-Ryan, A. E.; Trexler, E. T.; Hirsch, K. R.; Mock, M. G. Effects of pomegranate extract on blood flow and vessel diameter after high-intensity exercise in young, healthy adults. Eur J Sport Sci 2017, 17, 317-325, doi:10.1080/17461391.2016.1230892.
36. Louati, K.; Berenbaum, F. Fatigue in chronic inflammation—a link to pain pathways. Arthritis Res Ther 2015, 17, 254, doi:10.1186/s13075-015-0784-1.
37. Maes, M.; Twisk, F. N.; Kubera, M.; Ringel, K. Evidence for inflammation and activation of cell-mediated immunity in Myalgic Encephalomyelitis/Chronic Fatigue Syndrome (ME/CFS): increased interleukin-1, tumor necrosis factor-alpha, PMN-elastase, lysozyme and neopterin. J Affect Disord 2012, 136, 933-939, doi:10.1016/j.jad.2011.09.004.
38. Lee, C. H.; Giuliani, F. The Role of Inflammation in Depression and Fatigue. Front Immunol 2019, 10, 1696, doi:10.3389/fimmu.2019.01696.
39. Mastrogiovanni, F.; Mukhopadhya, A.; Lacetera, N.; Ryan, M. T.; Romani, A.; Bernini, R.; Sweeney, T. Anti-Inflammatory Effects of Pomegranate Peel Extracts on In Vitro Human Intestinal Caco-2 Cells and Ex Vivo Porcine Colonic Tissue Explants. Nutrients 2019, 11, doi:10.3390/nu11030548.
40. Barati Boldaji, R.; Akhlaghi, M.; Sagheb, M. M.; Esmaeilinezhad, Z. Pomegranate juice improves cardiometabolic risk factors, biomarkers of oxidative stress and inflammation in hemodialysis patients: a randomized crossover trial. J Sci Food Agric 2020, 100, 846-854, doi:10.1002/jsfa.10096.
41. Shishehbor, F.; Mohammad Shahi, M.; Zarei, M.; Saki, A.; Zakerkish, M.; Shirani, F.; Zare, M. Effects of Concentrated Pomegranate Juice on Subclinical Inflammation and Cardiometabolic Risk Factors for Type 2 Diabetes: A Quasi-Experimental Study. Int J Endocrinol Metab 2016, 14, e33835, doi:10.5812/ijem.33835.
42. Hosseini, B.; Saedisomeolia, A.; Wood, L. G.; Yaseri, M.; Tavasoli, S. Effects of pomegranate extract supplementation on inflammation in overweight and obese individuals: A randomized controlled clinical trial. Complement Ther Clin Pract 2016, 22, 44-50, doi:10.1016/j.ctcp.2015.12.003.
43. Fremont, M.; Coomans, D.; Massart, S.; De Meirleir, K. High-throughput 16S rRNA gene sequencing reveals alterations of intestinal microbiota in myalgic encephalomyelitis/chronic fatigue syndrome patients. Anaerobe 2013, 22, 50-56, doi:10.1016/j.anaerobe.2013.06.002.
44. Logan, A. C.; Venket Rao, A.; Irani, D. Chronic fatigue syndrome: lactic acid bacteria may be of therapeutic value. Med Hypotheses 2003, 60, 915-923, doi:10.1016/s0306-9877(03)00096-3.
45. Sheedy, J. R.; Wettenhall, R. E.; Scanlon, D.; Gooley, P. R.; Lewis, D. P.; McGregor, N.; Stapleton, D. I.; Butt, H. L.; KL, D. E. M. Increased d-lactic Acid intestinal bacteria in patients with chronic fatigue syndrome. In Vivo 2009, 23, 621-628.

1. A composition comprising pomegranate extract and a substance selected from the group consisting of: vitamin C and vitamin D.

2. A composition according to claim 1, comprising pomegranate extract, vitamin C and a vitamin of B group.

3. The composition according to claim 2, consisting of pomegranate extract, vitamin C, a vitamin of B group and optionally a substance selected from the group consisting of: zinc, iron and copper.

4. A composition according to claim 1, comprising pomegranate extract and vitamin D or pomegranate extract, vitamin C and vitamin D.

5. The composition according to claim 4, consisting of:
pomegranate extract and vitamin D or
pomegranate extract, vitamin C and vitamin D
and optionally a substance selected from the group consisting of: zinc, iron and copper.

6. The composition according to claim 1 wherein the pomegranate extract is present in an amount from 100 mg to 600 mg.

7. The composition according to claim 1 wherein the pomegranate extract is a dry extract.

8. The composition according to claim 1, where the pomegranate extract is characterized by:
a total polyphenol content between 15% w/w and 85% w/w, preferably between 15% w/w and 45% w/w, preferably 15 or 20%, and/or
an ellagitannin content between 5 and 75% w/w, preferably between 5 and 15% w/w, preferably comprised between 7 and 10%, and/or
a content of ellagic acid and derivatives comprised between 1% w/w and 25% w/w, preferably between 1% w/w and 15% w/w, preferably not less than 3%.

9. A composition according to claim 1 comprising pomegranate extract, complex of vitamins of B group and Vitamin C.

10. Composition according to claim 9 wherein the complex of vitamins of B group comprises: Vitamin B1, Vitamin B2, Vitamin B6, Vitamin B12, Pantothenic acid, Niacin, Biotin and Folate.

11. A composition according to claim 9 wherein said composition comprises in percentage referred to the total weight of the composition:
pomegranate extract 5-80%, preferably about 31.2% or 25%,
complex of vitamins of B group 1-10%, preferably about 2.4% or 4%,
Vitamin C 5-30%, preferably about 12.5% or 8%.

12. A composition according to claim 1 wherein said composition comprises in w/w percentages with respect to the total of the active ingredients: pomegranate extract 40-80%, preferably about 68%, more preferably 67.7%, the complex of vitamins of the B group 1-10%, preferably about 5%, vitamin C 5-40%, preferably about 27%.

13. A composition according to claim 1 comprising pomegranate extract and vitamin D, preferably said composition comprises in percentage referred to the total weight of the composition: pomegranate extract 5-20%, preferably about 6%, and vitamin D 0.0001-0.10%, preferably about 0.0005%.

14. A composition according to claim 1 comprising in w/w percentages with respect to the total of the active ingredients:
pomegranate extract 70-99.999%, preferably about 99.99%, and vitamin D 0.0001-30%, preferably about 0.01% or
pomegranate extract about 88% and vitamin D about 12%.

15. A composition according to claim 1 comprising pomegranate extract, vitamin D and vitamin C, preferably said composition comprises in percentage referred to the total weight of the composition: pomegranate extract 5-50%, preferably about 20%, vitamin D 0.0001-0.10%, preferably about 0.001%, vitamin C 5-30%, preferably about 2.6%.

16. The composition according to claim 1 where:
i. pomegranate extract is present in an amount from 100 mg to 600 mg, preferably 150, 250, 300, 500 or 600 mg and/or
ii. B group vitamins are present in the following quantities:
Vitamin B1: from 1.1 mg to 25 mg, preferably 5.5 mg and/or
Vitamin B2: from 1.4 mg to 25 mg, preferably 7 mg and/or
Vitamin B6: from 1.4 mg to 10 mg, preferably 4 mg and/or
Vitamin B12: from 2.5 mcg (micrograms) to 1000 mcg, preferably 0.0125 mg and/or
Niacin: 16 mg to 54 mg, preferably 16 mg and/or
Pantothenic acid: 6 mg to 18 mg, preferably 6 mg and/or
Biotin: from 50 mcg to 450 mcg, preferably 0.05 mg and/or
folic acid: from 200 mcg to 400 mcg, preferably 0.2 mg and/or
iii. vitamin C is present in an amount of from 40 to 1000 mg, preferably 80 or 100 or 200 mg and/or
iv. vitamin D is present in an amount of 5 to 50 mcg, preferably 5 or 10 or 25 or 50 mcg.

17. The composition according to claim 1 where:
i. pomegranate extract is present in an amount from 100 mg to 600 mg, preferably 150 or 250 or 300 or 500 or 600 mg and
ii. B group vitamins are present in the following quantities:
Vitamin B1: from 1.1 mg to 25 mg, preferably 5.5 mg and
Vitamin B2: from 1.4 mg to 25 mg, preferably 7 mg and
Vitamin B6: from 1.4 mg to 10 mg, preferably 4 mg and
Vitamin B12: from 2.5 mcg (micrograms) to 1000 mcg, preferably 0.0125 mg and
Niacin: 16 mg to 54 mg, preferably 16 mg and
Pantothenic acid: 6 mg to 18 mg, preferably 6 mg and
Biotin: from 50 mcg to 450 mcg, preferably 0.05 mg and
folic acid: from 200 mcg to 400 mcg, preferably 0.2 mg and
iii. vitamin C is present in an amount of from 40 to 1000 mg, preferably 80 or 100 or 200 mg.

18. The composition according to claim 1 wherein:
pomegranate extract is present in an amount from 100 mg to 600 mg, preferably 150 or 250 or 300 or 500 or 600 mg and
vitamin C is present in an amount of from 40 to 1000 mg, preferably 80 or 100 or 200 mg and
vitamin D is present in an amount of 5 to 50 mcg, preferably 5 or 10 or 25 or 50 mcg.

19. The composition according to claim 1 wherein:
pomegranate extract is present in an amount from 100 mg to 600 mg, preferably 150 or 250 or 300 or 500 or 600 mg and
vitamin D is present in an amount of 5 to 50 mcg, preferably 5 or 10 or 25 or 50 mcg.

20. The composition according to claim 1 where the pomegranate extract, vitamin C and vitamin D and the vitamins of B group are the only substances present in the composition which are active in activating the immune system and/or in increasing the immune defenses and/or the body's ability to recover from psycho-physical stress, in strengthening the immune system, reducing the period and intensity of colds and flu infections, preventing colds and flu and/or fighting colds and influenza, and/or preventing and/or treating upper respiratory tract infections, influenza virus infection, in convalescence after a seasonal illness or a surgery, and/or in the prevention and/or treatment of diseases or symptoms related to oxidative stress and/or in recovering from tiredness and exhaustion following an infection, or other cause and/or in countering and/or reducing fatigue or tiredness, preferably chronic fatigue.

21. Pharmaceutical composition comprising the composition according to claim 1 and at least one pharmaceutically acceptable excipient and/or vehicle.

22. A food supplement or product or drinking product comprising the composition according to claim 1.

23. (canceled)

24. A method of activating the immune system or in increasing the immune defenses and/or abilities of the body to recover from psycho-physical stress, reducing the period and intensity of colds and flu infections, preventing colds and flu and/or fighting colds and flu, and/or for use in the prevention and/or treatment of upper respiratory tract infections, influenza virus infection, in convalescence after seasonal illness or surgery, and/or for the prevention and/or treatment of oxidative stress-related conditions or symptoms and/or in recovery from tiredness and exhaustion following an infection, or other cause and/or in counteracting and/or reducing fatigue or tiredness, comprising administering an effective amount of a composition of claim 1 to subject in need of such treatment.

25. (canceled)

26. (canceled)
